(19) United States
(12) Patent Application Publication
(10) Pub. No.: US 2019/0358807 A1
(43) Pub. Date: Nov. 28, 2019

(54) ASSIST DEVICE

(71) Applicant: JTEKT CORPORATION, Osaka-shi (JP)

(72) Inventors: Hiromichi OHTA, Kariya-shi (JP); Kazuyoshi OHTSUBO, Chiryu-shi (JP)

(73) Assignee: JTEKT CORPORATION, Osaka-shi (JP)

(21) Appl. No.: 16/422,255

(22) Filed: May 24, 2019

(30) Foreign Application Priority Data
May 28, 2018 (JP) 2018-101841

(51) Int. Cl. B25J 9/00; A61H 3/00; A61B 5/00; A61H 1/02

(52) U.S. Cl. CPC B25J 9/0006; A61H 3/008; A61B 5/6831; A61H 2201/5061; A61H 2201/165; A61H 2201/5069; A61H 2201/1628; A61H 1/0244

(57) ABSTRACT

An assist device includes a body-worn component that is worn at least around a hip of a user, a thigh unit that is mounted on the body-worn component and each thigh of the user, a torque generation unit having an actuator configured to generate assist torque, a control box disposed above the hip of the user on the body-worn component, the control box accommodating the torque generation unit and a controller configured to control the torque generation unit, and a power transmission unit configured to transmit assist torque generated in the control box to the thigh unit. The assist device reduces a load on a lower back of the user by assisting the user with a movement of each thigh relative to the lower back.

### INCORPORATION BY REFERENCE

The disclosure of Japanese Patent Application No. 2018-101841 filed on May 28, 2018 including the specification, drawings and abstract is incorporated herein by reference in its entirety.

### BACKGROUND

### 1. Technical Field

The disclosure relates to an assist device that assists a user with movements of an intended body part.

### 2. Description of Related Art

For example, Japanese Unexamined Patent Application Publication No. 2017-154210 (JP 2017-154210 A) describes a motion assist device. The motion assist device is worn on a body of a user, and reduces a load on the lower back of the user by assisting the user with the motion to straighten up at the time of lifting up baggage. The motion assist device includes a waist belt, a right shoulder belt, a left shoulder belt, a back plate, a lower back plate, a right body part, a right thigh pressing part, a left body part, a left thigh pressing part, and other parts. The waist belt is worn around the waist of the user. One end of the right shoulder belt is connected to the right front of the waist belt via the right shoulder of the user. The other end of the right shoulder belt is connected to the right rear of the waist belt. One end of the left shoulder belt is connected to the left front of the waist belt via the left shoulder of the user. The other end of the left shoulder belt is connected to the left rear of the waist belt. The right body part accommodates a right drive unit that generates assist torque for assisting with the movement of the right thigh through the right thigh pressing part. The right body part is connected to the right side (right hip portion) of the waist belt. The left body part accommodates a left drive unit that generates assist torque for assisting with the movement of the left thigh through the left thigh pressing part. The left body part is connected to the left side (left hip portion) of the waist belt. The back plate is disposed in the region of the back between both shoulders of the user. The back plate is connected to the right body part and the left body part by a right side frame and a left side frame. A back frame is disposed between the right side frame and the left side frame. The lower back plate is connected to the back frame. Each of the right body part and the left body part accommodates a motor, a power supply, a sensor, and other parts as the drive unit.

### SUMMARY

In the motion assist device described in JP 2017-154210 A, the motor, the power supply, the sensor, and other parts are accommodated in each of the right body part disposed at the right hip region of the user and the left body part disposed at the left hip region of the user. Therefore, the right body part and the left body part are remarkably bulky and heavy. Since the right body part and the left body part are bulky, there is a large projection from each of the right and left sides of the user. Therefore, when the user lifts up baggage, movements of the arms of the user may interfere with the right body part or the left body part, with the result that appropriate motion assistance may not be performed. In addition, since each of the right body part and the left body part includes the motor, the power supply, the sensor, and other parts accommodated in a large case and is remarkably heavy, even when each of the right body part and the left body part is able to assist with the movement of a corresponding one of the thighs, its weight may dilute the effect of reducing a load on the lower back of the user.

The disclosure is made in view of the above situations, and provides a further lightweight assist device that does not interfere with the movement of each arm of a user when the user performs lift-up motion or bring-down motion.

An aspect of the present disclosure is an assist device that reduces a load on a lower back of a user by assisting the user with a movement of each thigh of the user relative to the lower back. The assist device includes: a body-worn component worn at least around a hip of the user; a thigh unit mounted on the body-worn component and each thigh of the user, the thigh unit is configured to transmit assist torque for assisting the user with a movement of the thigh relative to the lower back or a movement of the lower back relative to the thigh; a torque generation unit having an actuator configured to generate the assist torque; a control box disposed above the hip of the user on the body-worn component, the control box accommodates the torque generation unit and a controller configured to control the torque generation unit; and a power transmission unit configured to transmit assist torque generated in the control box to the thigh unit.

With the above assist device, since the torque generation unit and the controller are accommodated in the control box disposed above the hip of the user, no torque generation unit (such as motor) or battery is disposed at a right hip region or left hip region of the user. Therefore, there is no bulky parts that interfere with movements of arms of the user during lift-up motion or bring-down motion at the right hip region or left hip region of the user. In addition, the torque generation unit for assisting a right thigh and the torque generation unit for assisting a left thigh are collectively accommodated in the single control box, so a case and other parts can be shared. Hence, the weight is further reduced.

In above assist device, the power transmission unit may include a cable and a tension adjustment part configured to adjust tension of the cable.

With the above assist device, the power transmission unit is simple and lightweight.

In above assist device: the body-worn component may include a lower back support part worn around the hip of the user; the thigh unit may include a right thigh unit worn on a right thigh of the user and a left thigh unit worn on a left thigh of the user; the right thigh unit may be mounted on the lower back support part via a right hip base, the right hip base may be connected to a right hip portion of the lower back support part; the left thigh unit may be mounted on the lower back support part via a left hip base, the left hip base may be connected to a left hip portion of the lower back support part; the power transmission unit may include a right frame and a left frame, the right hip base and the control box may be connected via the right frame, the left hip base and the control box may be connected via the left frame; and each of the right frame and the left frame may have less stiffness in a right and left direction with respect to the user wearing the assist device than stiffness in a front and rear direction with respect to the user, each of the right frame and the left frame may configured to elastically deform so as to curve in the right and left direction for a width of the hip of the user.

With the above assist device, simple and non-bulky configuration at the right hip region and the left hip region are achieved. In addition, adjustment for the width of the hip of the user is achieved by elastic deformation of the right frame and left frame, so no special hip width adjustment mechanism needs to be provided. Hence, size reduction and weight reduction are further improved.

In above assist device: the thigh unit may include a thigh worn portion having a surface that contacts with the thigh of the user and a thigh belt wound around the thigh of the user; the thigh worn portion may have a plurality of slits along a thigh extending direction that is a direction in which the thigh of the user extends; the thigh belt may be inserted through the slits; and a length of the slits in the thigh extending direction may be greater than a length of the thigh belt in the thigh extending direction such that the thigh belt inserted through the slits is slidable along the thigh extending direction of the slits.

With the above assist device, since the thigh belt fixedly wound around the thigh of the user is slidable in the thigh extending direction along the slits in response to a pivot angle of the thigh relative to the upper body of the user, no special slide mechanism needs to be provided for the thigh worn portion. Hence, size reduction and weight reduction are further improved.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a perspective view illustrating an example of the overall configuration of an assist device of a first embodiment;

FIG. 2 is a perspective view illustrating an example of the overall configuration of an assist device of which part of a jacket part is changed from that of the assist device of FIG. 1;

FIG. 3 is an exploded perspective view of the assist device shown in FIG. 1;

FIG. 4 is a perspective view illustrating an example of the appearance of a body-worn component in the assist device shown in FIG. 1;

FIG. 5 is a perspective view illustrating an example of the appearance of actuator units in the assist device shown in FIG. 1;

FIG. 6 is a perspective view illustrating an example of the appearance of a frame part that is an element of the body-worn component;

FIG. 7 is a perspective view illustrating an example of the appearance of a lower back support part that is an element of the body-worn component;

FIG. 8 is an expansion plan illustrating an example of the structure of the lower back support part;

FIG. 9 is a perspective view illustrating an example of the appearance of a backpack part (and the frame part) that is an element of the body-worn component;

FIG. 10 is a perspective view illustrating an example of the appearance of a state where the jacket part that is an element of the body-worn component is connected to the backpack part and the frame part;

FIG. 11 is an expansion plan illustrating an example of the structure of the jacket part;

FIG. 12 is a perspective view of the actuator unit (right) in the assist device shown in FIG. 1;

FIG. 13 is a perspective view illustrating another example of the actuator unit (right) shown in FIG. 12;

FIG. 14 is a perspective view illustrating a structure around a thigh worn portion (body holding portion);

FIG. 15 is a view illustrating an example in which a below-knee belt is added to the body holding portion shown in FIG. 14;

FIG. 16 is a view illustrating an example in which a third joint of the thigh worn portion (body holding portion) in the actuator unit (right) shown in FIG. 13 is disposed on the front of the thigh of a user;

FIG. 17 is a view illustrating an example in which the third joint of the thigh worn portion (body holding portion) in the actuator unit (right) shown in FIG. 13 is disposed on the side that is the outer side of the thigh of the user;

FIG. 18 is a view illustrating an example in which the third joint of the thigh worn portion (body holding portion) in the actuator unit (right) shown in FIG. 13 is disposed on the back of the thigh of the user;

FIG. 19 is an exploded perspective view illustrating an example of the internal structure of the actuator unit;

FIG. 20 is a cross-sectional view illustrating an example of the internal structure of the actuator unit;

FIG. 21 is a view illustrating an erect state where the user wearing the assist device squares up;

FIG. 22 is a view illustrating a state where the user bent the upper body forward from the state shown in FIG. 21 and the frame part and other parts pivoted around an assumed pivot axis;

FIG. 23 is a view illustrating an example of the appearance of an operating unit;

FIG. 24 is a view illustrating inputs and outputs of a controller;

FIG. 25 is a view illustrating how operation mode, gain, and rate of increase are changed (adjusted) through the operating unit;

FIG. 26 is a control block diagram for controlling the actuator unit by the controller;

FIG. 27 is a flowchart illustrating the overall procedure based on the control block diagram shown in FIG. 26;

FIG. 28 is a flowchart illustrating the details of a process of adjustment determination, input processing, and torque change calculation in S100 in the flowchart shown in FIG. 27;

FIG. 29 is a flowchart illustrating the details of a process of motion type determination in S200 in the flowchart shown in FIG. 27;

FIG. 30 is a flowchart illustrating the details of a process of bring-down (right) in SD000R in the flowchart shown in FIG. 27;

FIG. 31 is a view illustrating a scene of bring-down work of the user;

FIG. 32 is a graph illustrating an example of user torque change amount-assist amount characteristics;

FIG. 33 is a graph illustrating an example of forward lean angle-bring-down torque limit characteristics;

FIG. 34 is a timing chart illustrating a state of a change in forward lean angle and a change in bring-down assist torque to a time when the user performs bring-down work;

FIG. 35 is a flowchart illustrating the details of a process of lift-up in SU000 in the flowchart shown in FIG. 27;

FIG. 36 is a state transition diagram illustrating the details of a process of motion status determination in SS000 in the flowchart shown in FIG. 35;

FIG. 37 is a graph illustrating a state of a change in forward lean angle and a change in lift-up assist torque to a transition of motion status when the user performs lift-up work;

FIG. 38 is a flowchart illustrating the details of a process of determination as to whether to shift the rate of increase (right) in SS100R in the flowchart shown in FIG. 35;

FIG. 39 is a timing chart illustrating an example of time-shift lower limit characteristics and an example of time-shift upper limit characteristics;

FIG. 40 is a table illustrating an example of rate of increase-transition time characteristics;

FIG. 41 is a timing chart illustrating an example of time-assist amount characteristics;

FIG. 42 is a flowchart illustrating the details of a process of assist torque calculation (right) in SS170R in the flowchart shown in FIG. 35;

FIG. 43 is a timing chart illustrating an example of time-lift-up torque characteristics and forward lean angle-lift-up maximum torque characteristics;

FIG. 44 is a table illustrating an example of gain-attenuation coefficient characteristics;

FIG. 45 is a graph illustrating an example of assist ratio-torque attenuation rate characteristics;

FIG. 46 is a perspective view illustrating an example of the overall configuration of an assist device of a second embodiment;

FIG. 47 is an exploded perspective view of the assist device shown in FIG. 46;

FIG. 48 is a perspective view illustrating an example of the appearance of a body-worn component in the assist device shown in FIG. 46;

FIG. 49 is a view (side view) illustrating a power transmission unit that transmits power from a right drive pulley of a backpack part to a right driven pulley of a right hip base part;

FIG. 50 is a cross-sectional view taken along the line A-A in FIG. 49;

FIG. 51 is a view (front view) illustrating the power transmission unit that transmits power from the right drive pulley of the backpack part to the right driven pulley of the right hip base;

FIG. 52 is a view illustrating the configuration of torque generation units accommodated in the backpack part;

FIG. 53 is an exploded perspective view illustrating an example of the configuration of the torque generation unit;

FIG. 54 is a view illustrating an example of the appearance of a thigh unit, thigh worn portion, and thigh belt; and

FIG. 55 is a view illustrating an example in which the power transmission unit made up of the pulleys and cables shown in FIG. 49 is replaced with a power transmission unit made up of a parallel linkage.

### DETAILED DESCRIPTION OF EMBODIMENTS

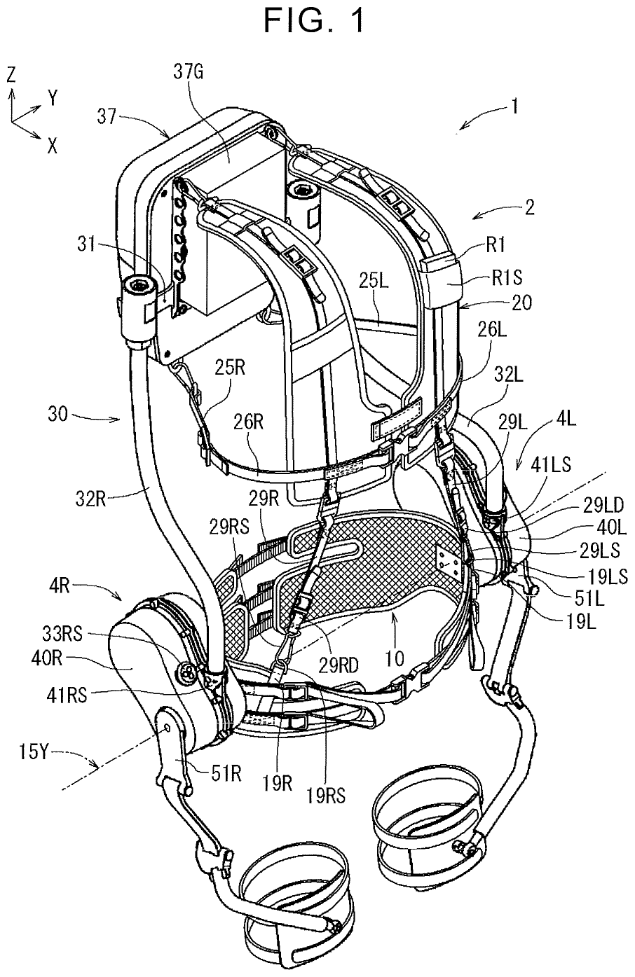

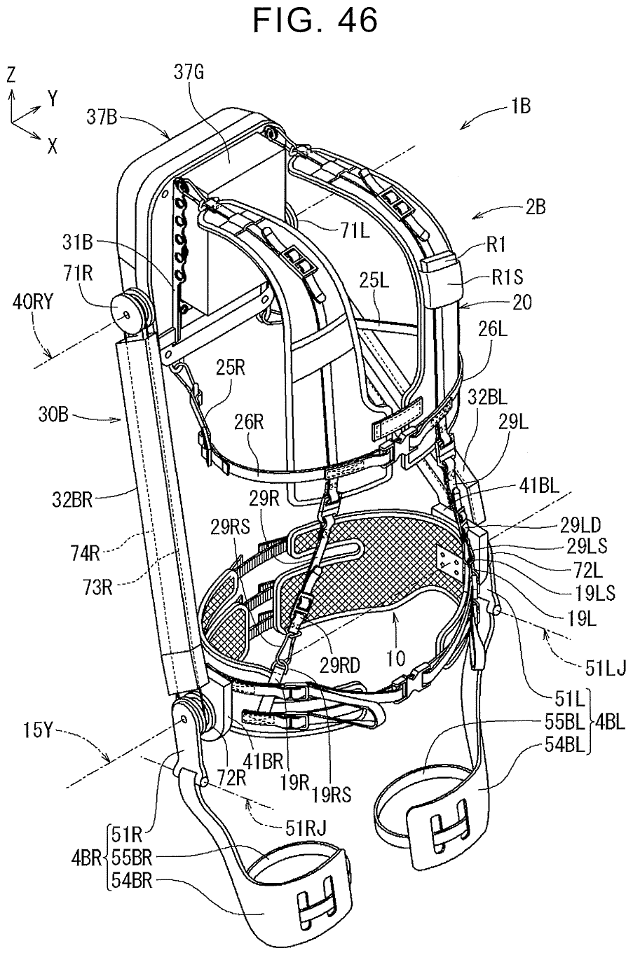

Hereinafter, the overall structure of an assist device 1 (see FIG. 1) of a first embodiment, the procedure of a controller of the assist device, and the like, will be described with reference to FIG. 1 to FIG. 45, and the overall structure of an assist device 1B (see FIG. 46) of a second embodiment will be described with reference to FIG. 46 to FIG. 55. The procedure of a controller of the assist device 1B of the second embodiment is similar to the procedure of the controller of the assist device 1 of the first embodiment, so the description thereof is omitted. The assist device 1 of the first embodiment is an example of the structure in which torque generation units, such as electric motors, are respectively worn at the right hip region and left hip region of a user. The assist device 1B of the second embodiment is an example of the structure in which torque generation units, such as electric motors, are accommodated in a backpack part 37B (see FIG. 46, which may be regarded as a control box) disposed above the hip of a user.

Hereinafter, the overall structure of the assist device 1 will be described with reference to FIG. 1 to FIG. 25. The assist device 1, for example, assists the user hi pivoting the thighs relative to the lower back (or the lower back relative to the thighs) when the user lifts up baggage (or when the user brings down baggage) or assists the user in pivoting the thighs relative to the lower back when the user walks. The X-axis, Y-axis, and Z-axis in the drawings are perpendicular to one another. When viewed from the user wearing the assist device, the X-axis direction may be regarded as a forward direction, the Y-axis direction may be regarded as a leftward direction, and the Z-axis direction may be regarded as an upward direction.

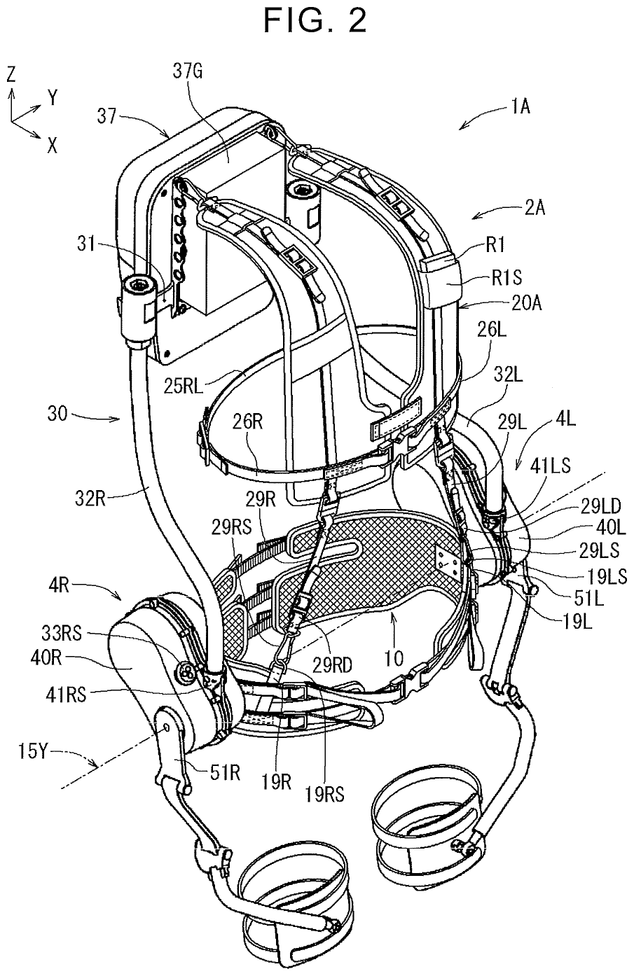

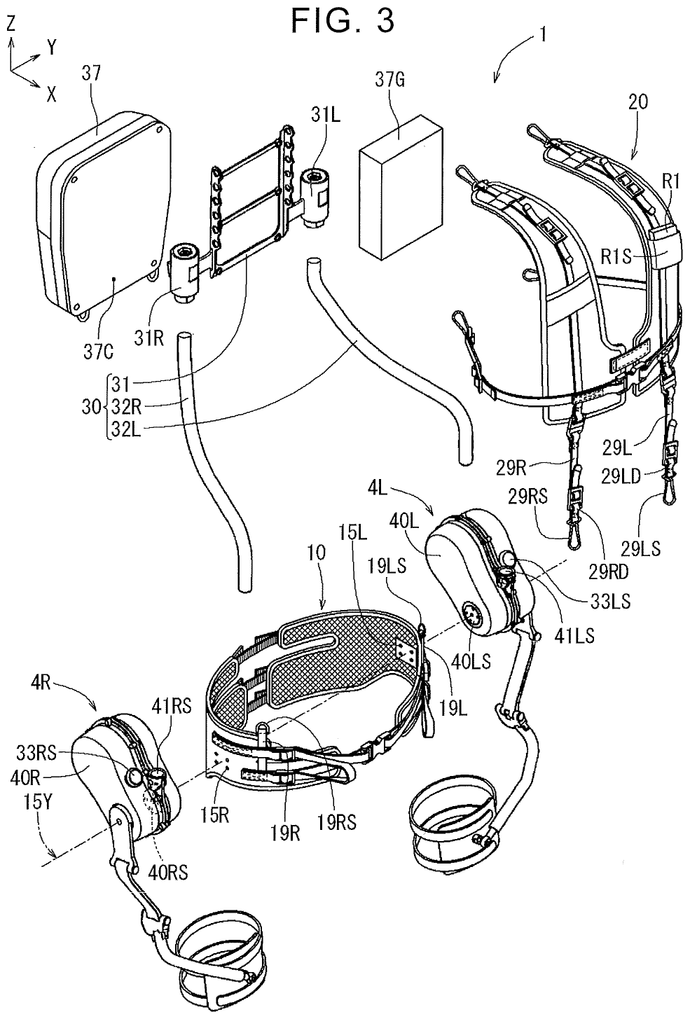

FIG. 1 shows the overall appearance of the assist device 1. FIG. 2 shows the overall appearance of an assist device 1A of which a right armpit belt 25R and a left armpit belt 25L in FIG. 1 are replaced with a close contact belt 25RL. The assist device 1A (a body-worn component 2A and a jacket part 20A) shown in FIG. 2 differs from the assist device 1 (a body-worn component 2 and a jacket part 20) shown in FIG. 1 only in the close contact belt 25RL. Therefore, hereinafter, the assist device 1 shown in FIG. 1 will be described, and the description of the assist device 1A shown in FIG. 2 is omitted. FIG. 3 shows an exploded perspective view of the assist device 1 shown in FIG. 1.

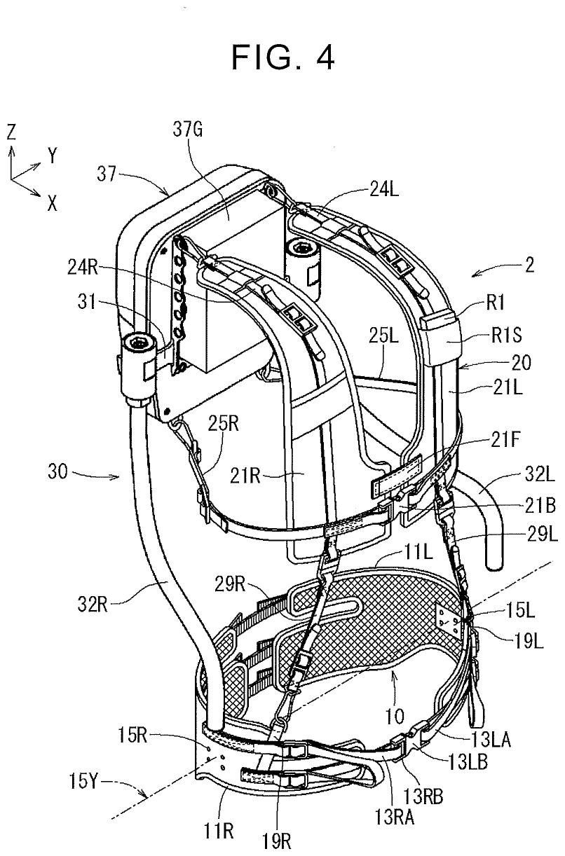

As shown by the exploded perspective view of FIG. 3, the assist device 1 is made up of a lower back support part 10, the jacket part 20, a frame part 30, a backpack part 37, a cushion 37G, a right actuator unit 4R, a left actuator unit 4L, and other parts. The body-worn component 2 (see FIG. 4) is made up of the lower back support part 10, the jacket part 20, the frame part 30, the backpack part 37, and the cushion 37G. An actuator unit 4 is made up of the right actuator unit 4R and the left actuator unit 4L. The assist device 1 also includes an operating unit R1 (so-called remote control unit) and a holder R1S. The operating unit R1 is a unit that the user adjusts operation mode (bring-down assist mode, lift-up assist mode, or other modes), the gain of assist torque, and the rate of increase in assist torque or checks the adjusted statuses or other information. The holder R1S holds the operating unit R1.

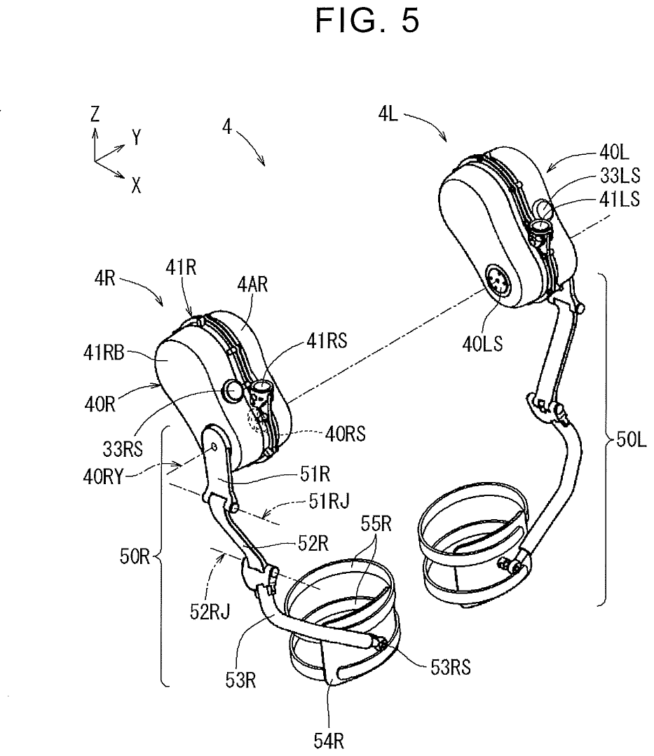

The body-worn component 2 (see FIG. 4) is worn at least around the hip of the user. Each of the right actuator unit 4R and the left actuator unit 4L (see FIG. 5) is mounted on the body-worn component 2 and also worn on the thigh of the user, and assists the user with the movement of the thigh relative to the lower back or the movement of the lower back relative to the thigh. Hereinafter, the body-worn component 2 and the actuator unit 4 will be described in turn.

As shown in FIG. 3 and FIG. 4, the body-worn component 2 includes the lower back support part 10, the jacket part 20, the frame part 30, the backpack part 37, and the cushion 37G. The lower back support part 10 is worn around the hip of the user. The jacket part 20 is worn around the shoulders and chest of the user. The jacket part 20 is connected to the frame part 30. The backpack part 37 and the cushion 37G are mounted on the frame part 30. The frame part 30 is disposed around the back and shoulders of the user.

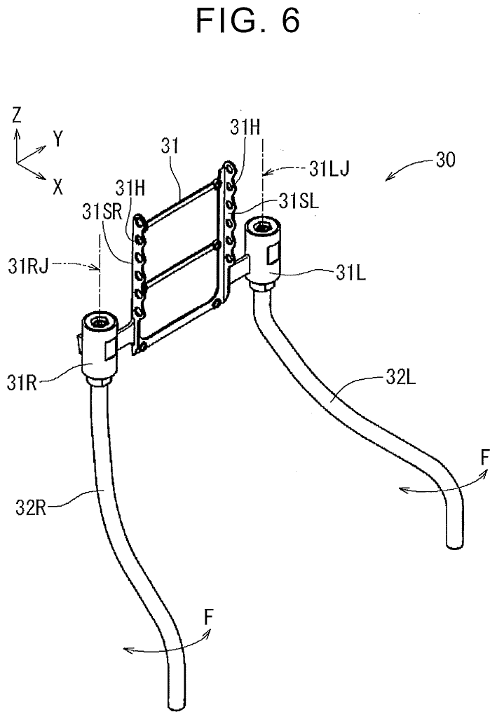

As shown in FIG. 3 and FIG. 6, the frame part 30 includes a main frame 31, a right sub-frame 32R, a left sub-frame 32L, and other parts. As shown in FIG. 6, the main frame 31 has support bodies 31SR, 31SL, a connection portion 31R (right pivot shaft portion), and a connection portion 31L (left pivot shaft portion). A plurality of belt connection holes 31H is disposed in each of the support bodies 31SR, 31SL in an upper and lower direction. One end (upper end) of the right sub-frame 32R is connected to the connection portion 31R. One end (upper end) of the left sub-frame 32L is connected to the connection portion 31L. The connection portion 31R is a so-called cylindrical damper. The connection portion 31R includes coaxially disposed inner and outer cylinders and a cylindrical elastic body disposed between the inner cylinder and the outer cylinder. The outer cylinder is fixed to the main frame 31. One end (upper end) of the right sub-frame 32R is fixed to the inner cylinder. Similarly, the outer cylinder of the connection portion 31L is fixed to the main frame 31, and one end (upper end) of the left sub-frame 32L is fixed to the inner cylinder. Thus, the right sub-frame 32R is pivotable about a pivot axis 31RJ, and the left sub-frame 32L is pivotable about a pivot axis 31LJ.

As shown in FIG. 1, the lower end of the right sub-frame 32R is connected (fixed) to a connection portion 41RS of the right actuator unit 4R, and the lower end of the left sub-frame 32L is connected (fixed) to a connection portion 41LS of the left actuator unit 4L.

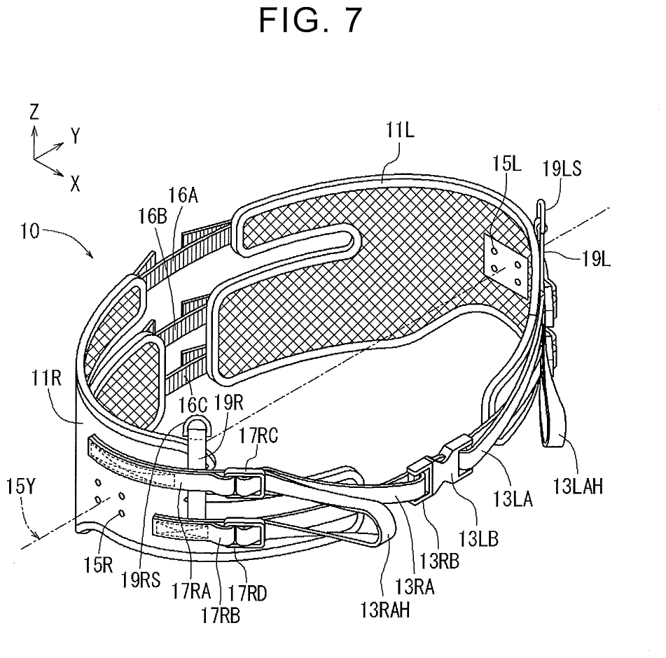

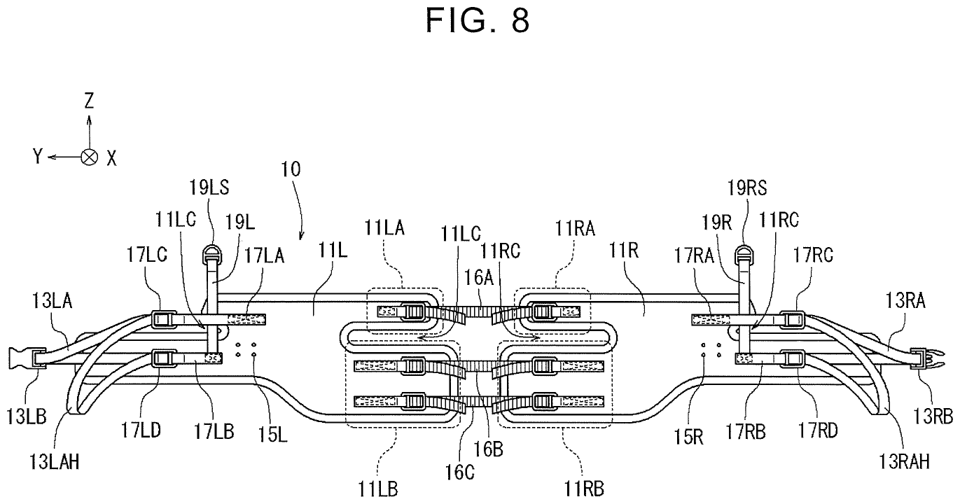

As shown in FIG. 7 and FIG. 8, the lower back support part 10 includes a right hip worn portion 11R and a left hip worn portion 11L. The right hip worn portion 11R is worn around the hip on the right side of the body of the user. The left hip worn portion 11L is worn around the hip on the left side of the body of the user. As shown in FIG. 8, the right hip worn portion 11R and the left hip worn portion 11L are connected by a back hip belt 16A, an upper buttock belt 16B, and a lower buttock belt 16C.

As shown in FIG. 1 and FIG. 3, the lower back support part 10 includes a coupling belt 19R and a coupling belt 19L. The coupling belt 19R has a coupling ring 19RS that is coupled to a coupling portion 29RS of the jacket part 20. The coupling belt 19L has a coupling ring 19LS that is coupled to a coupling portion 29LS of the jacket part 20. As shown in FIG. 3, the lower back support part 10 has mounting holes 15R and mounting holes 15L at locations that intersect with an assumed pivot axis 15Y. The mounting holes 15R are provided for connection with the coupling portion 40RS of the right actuator unit 4R. The mounting holes 15L are provided for connection with the coupling portion 40LS of the left actuator unit 4L.

As shown in FIG. 8, a cutout 11RC is formed at a location on the back side of the user in the right hip worn portion 11R. The cutout 11RC separates the right hip worn portion 11R into a right lower back portion 11RA and a right buttock portion 11RB. A cutout 11LC is formed at a location on the back side of the user in the left hip worn portion 11L. The cutout 11LC separates the left hip worn portion 11L into a left lower back portion 11LA and a left buttock portion 11LB.

As shown in FIG. 7 and FIG. 8, the lower back support part 10 includes various belts with adjustable length and other parts for close contact with the hip of the user without a slip. The various belts and other parts include a right hip fastening belt 13RA, a hip belt holding member 13RB (hip buckle), a left hip fastening belt 13LA, a hip belt holding member 13LB (hip buckle), a right upper pelvis belt 17RA, a right lower pelvis belt 17RB, a left upper pelvis belt 17LA, a left lower pelvis belt 17LB, a right upper belt holding member 17RC (right upper adjuster), a right lower belt holding member 17RD (right lower adjuster), a pulling portion 13RAH, a left upper belt holding member 17LC (left upper adjuster), a left lower belt holding member 17LD (left lower adjuster), a pulling portion 13LAH, and other parts.

As shown in FIG. 3, the backpack part 37 is mounted on the main frame 31 that is the upper end of the frame part 30. A right shoulder belt 24R, the right armpit belt 25R, a left shoulder belt 24L, and the left armpit belt 25L of the jacket part 20 are connected to the main frame 31 or the backpack part 37.

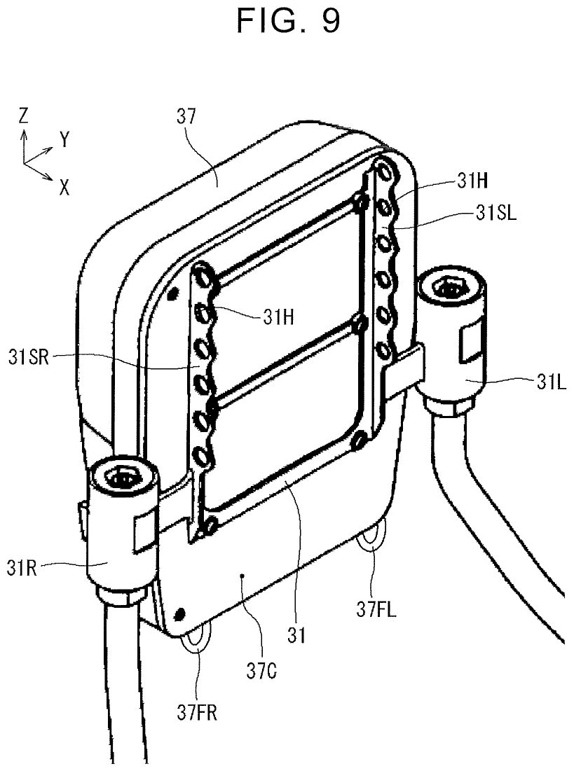

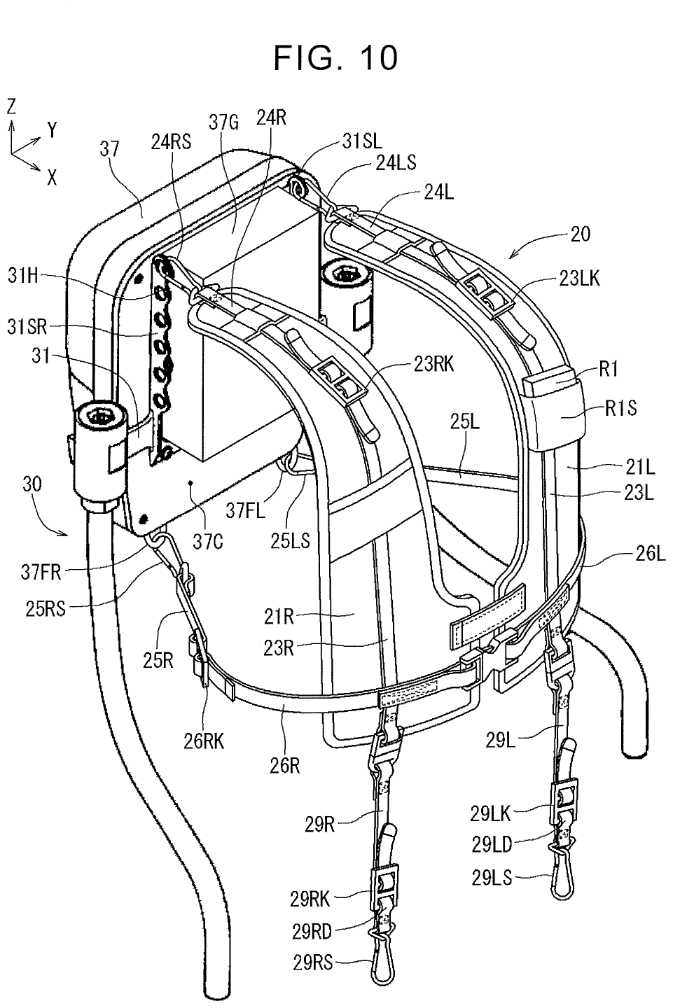

As shown in FIG. 9 and FIG. 10, the backpack part 37 has a simple box shape, and accommodates a controller, a power supply unit, a communication unit, and other parts. As shown in FIG. 9, the backpack part 37 includes a back stopper 37C at the main frame 31 side. The back stopper 37C is fixed to the main frame 31. The support bodies 31SR, 31SL are provided at locations that face both shoulders on the back side of the user in the main frame 31. The plurality of belt connection holes 31H (which may be regarded as belt connection portions) are disposed in each of the support bodies 31SR, 31SL in the upper and lower direction. That is, the plurality of belt connection holes 31H (belt connection portions) is provided so that the position of the jacket part 20 in the height direction relative to the frame part 30 is adjustable for the body size of the user. Therefore, the level of the jacket part 20 is adjustable to an appropriate position for the size of the user.

Even when the upper body of the user leans forward, the actuator units 4R, 4L that output assist torque are appropriately supported when the cushion 37G (or the back stopper 37C) that contacts with the back is elongated in a direction from the shoulders toward the lower back of the user. Even when the upper body of the user leans rightward or leftward, the cushion 37G (or the back stopper 37C) contacts with the flexion center of the back of the user, so the actuator units 4R, 4L that output assist torque are further appropriately supported (support stiffness increases).

As shown in FIG. 10, a belt connection portion 24RS of the right shoulder belt 24R is connected to any one of the belt connection holes 31H (belt connection portions) of the support body 31SR. Similarly, as shown in FIG. 10, a belt connection portion 24LS of the left shoulder belt 24L is connected to any one of the belt connection holes 31H (belt connection portions) of the support body 31SL. The support bodies 31SR, 31SL may be provided in the backpack part 37.

As shown in FIG. 9 and FIG. 10, belt connection portions 37FR, 37FL are provided at right and left sides of the lower end of the backpack part 37. As shown in FIG. 10, the belt connection portion 25RS of the right armpit belt 25R is connected to the belt connection portion 37FR. Similarly, as shown in FIG. 10, the belt connection portion 25LS of the left armpit belt 25L is connected to the belt connection portion 37FL. The belt connection portions 37FR, 37FL may be provided in the main frame 31.

As shown in FIG. 4, the jacket part 20 includes a right chest worn portion 21R and a left chest worn portion 21L. The right chest worn portion 21R is worn on the chest on the right side of the body of the user. The left chest worn portion 21L is worn on the chest on the left side of the body of the user. The right chest worn portion 21R is connected to the left chest worn portion 21L by, for example, a hook-and-loop fastener 21F and a buckle 21B, which makes it easy for the user to put on or off the jacket part 20.

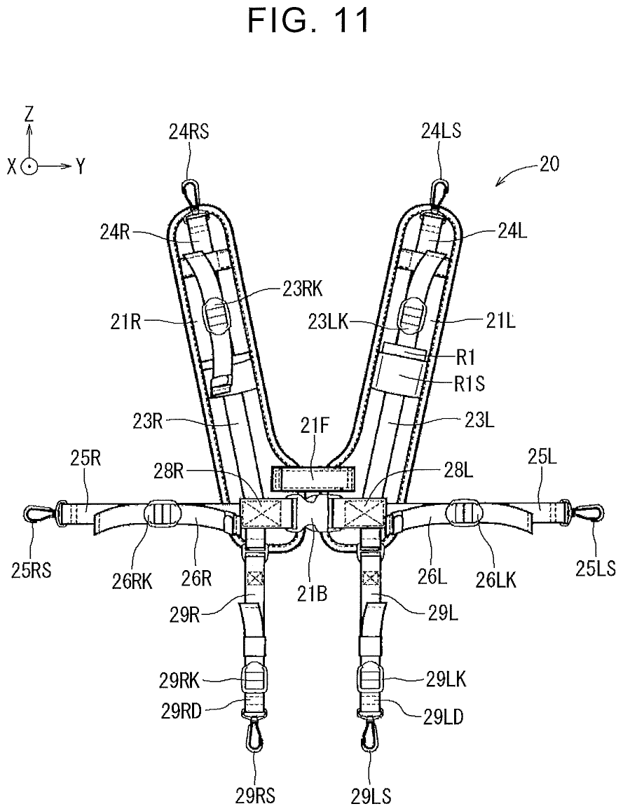

The right chest worn portion 21R includes the right shoulder belt 24R, the belt connection portion 24RS, the right armpit belt 25R, and the belt connection portion 25RS. The right shoulder belt 24R and the belt connection portion 24RS are connected to any one of the belt connection holes 31H of the main frame 31 (or the backpack part 37). The right armpit belt 25R and the belt connection portion 25RS are connected to the belt connection portion 37FR of the backpack part 37 (or the main frame 31). The left chest worn portion 21L includes the left shoulder belt 24L, the belt connection portion 24LS, the left armpit belt 25L, and the belt connection portion 25LS. The left shoulder belt 24L and the belt connection portion 24LS are connected to any one of the belt connection holes 31H of the main frame 31 (or the backpack part 37). The left armpit belt 25L and the belt connection portion 25LS are connected to the belt connection portion 37FL of the backpack part 37 (or the main frame 31). As shown in FIG. 11, the right chest worn portion 21R includes a coupling belt 29R and the coupling portion 29RS for coupling with the right hip worn portion 11R, and the left chest worn portion 21L includes a coupling belt 29L and the coupling portion 29LS for coupling with the left hip worn portion 11L.

As shown in FIG. 10 and FIG. 11, the jacket part 20 includes various belts with adjustable length and other parts for close contact with the chest of the user without a slip. The various belts and other parts include a fixing portion 28R, a fixing portion 28L, a right shoulder belt 23R, a right shoulder belt holding member 23RK (right shoulder adjuster), a left shoulder belt 23L, a left shoulder belt holding member 23LK (left shoulder adjuster), a right armpit belt 26R, a right armpit belt holding member 26RK (right armpit adjuster), a left armpit belt 26L, a left armpit belt holding member 26LK (left armpit adjuster), and other parts.

FIG. 5 shows the appearance of the right actuator unit 4R and left actuator unit 4L shown in FIG. 3. The right actuator unit 4R and the left actuator unit 4L are bilaterally symmetric, so the description of the left actuator unit 4L is omitted hereinafter.

As shown in FIG. 5, the right actuator unit 4R includes the torque generation unit 40R and an output link 50R that is a torque transmission unit. The torque generation unit 40R includes an actuator base part 41R, a cover 41RB, and a coupling base 4AR. As shown in FIG. 5, the output link 50R is worn on an intended body part (in this case, thigh), and pivots about a joint (in this case, hip joint) of the intended body part (in this case, thigh). Assist torque that assists in pivoting the intended body part via the output link 50R is generated by the electric motor (actuator) in the torque generation unit 40R.

The output link 50R includes an assist arm 51R (which may be regarded as a first link), a second link 52R, a third link 53R, and a thigh worn portion 54R (which may be regarded as a body holding portion). The assist arm 51R pivots about a pivot axis 40RY by the resultant torque of assist torque generated by the electric motor in the torque generation unit 40R and user torque generated by the movement of the thigh of the user. One end of the second link 52R is connected to the distal end of the assist arm 51R so as to be pivotable about a pivot axis 51RJ. One end of the third link 53R is connected to the other end of the second link 52R so as to be pivotable about a pivot axis 52RJ. The thigh worn portion 54R is connected to the other end of the third link 53R via a third joint 53RS (in this case, spherical joint).

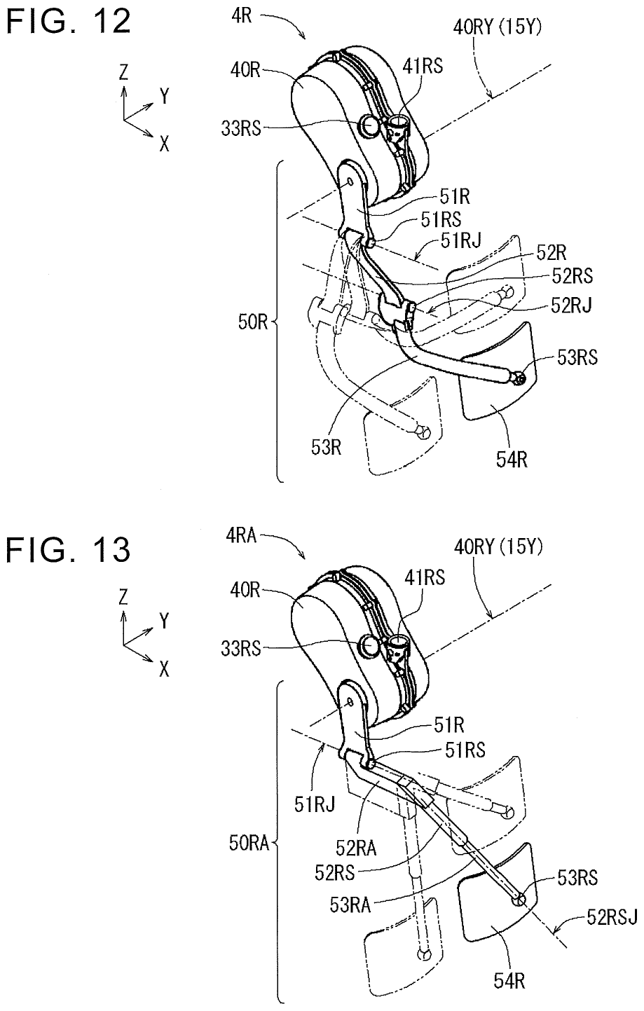

Next, the details of a link mechanism of the right actuator unit 4R will be described with reference to FIG. 5, and FIG. 12 to FIG. 18. As examples of the link mechanism, an example of the output link 50R shown in FIG. 12 and an example of an output link 50RA shown in FIG. 13 will be described.

The output link 50R shown in FIG. 12 is made up of a plurality of coupling members in a manner such that the assist arm 51R (which may be regarded as the first link), the second link 52R, the third link 53R, and the thigh worn portion 54R (which may be regarded as the body holding portion) are coupled by joints. The thigh worn portion 54R shown in FIG. 12 does not show a thigh belt 55R shown in FIG. 14.

One end of the second link 52R is coupled to the distal end of the assist arm 51R by a first joint 51RS so as to be pivotable about the pivot axis 51RJ. The first joint 51RS has a coupling structure with one degree of freedom such that the second link 52R is pivotable about the pivot axis 51RJ relative to the assist arm 51R.

One end of the third link 53R is coupled to the other end of the second link 52R by a second joint 52RS so as to be pivotable about the pivot axis 52RJ. The second joint 52RS has a coupling structure with one degree of freedom such that the third link 53R is pivotable about the pivot axis 52RJ relative to the second link 52R.

The other end of the third link 53R is coupled to the thigh worn portion 54R by the third joint 53RS (spherical joint in the example of FIG. 14). Therefore, the third joint 53RS between the third link 53R and the thigh worn portion 54R (body holding portion) has a coupling structure with three degrees of freedom. From above, the total number of degrees of freedom of the output link 50R shown in FIG. 12 is 1+1+3=5.

The total number of degrees of freedom of the output link 50R just needs to be greater than or equal to three. For example, as shown in FIG. 14, the third joint 53RS may be configured such that the thigh worn portion 54R is pivotable about the pivot axis 53RJ relative to the other end of the third link 53R. In the example of FIG. 14, the third joint 53RS has a coupling structure with one degree of freedom such that the thigh worn portion 54R is pivotable about the pivot axis 53RJ relative to the third link 53R. Therefore, since the number of degrees of freedom of the first joint 51RS is one and the number of degrees of freedom of the second joint 52RS is one, the total number of degrees of freedom of the output link in this case is 1+1+1=3. A stopper that limits the pivotable range of the second link or third link may be provided.

As shown in FIG. 14, the body holding portion is made up of the thigh worn portion 54R and the thigh belt 55R. The thigh worn portion 54R is coupled to the third link 53R and is worn on the thigh of the user. The thigh belt 55R is stretchable and provided at the thigh worn portion 54R so as to wrap the thigh of the user. The thigh belt 55R is made of a stretchable elastic material. One-end side of the thigh belt 55R is fixed to the thigh worn portion 54R. The other-end side of the thigh belt 55R serves as a hook-and-loop fastener 55RM. In addition, a hook-and-loop fastener 54RM is provided at a location that faces the other-end side of the thigh belt 55R on the thigh worn portion 54R.

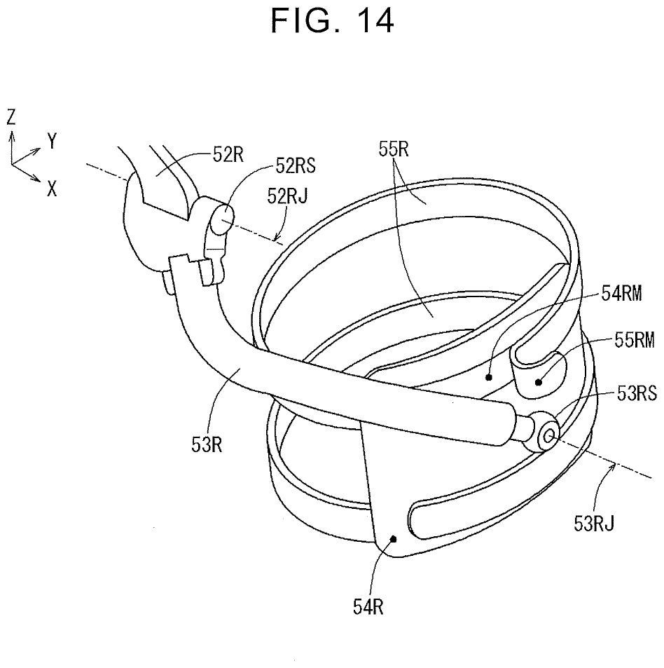

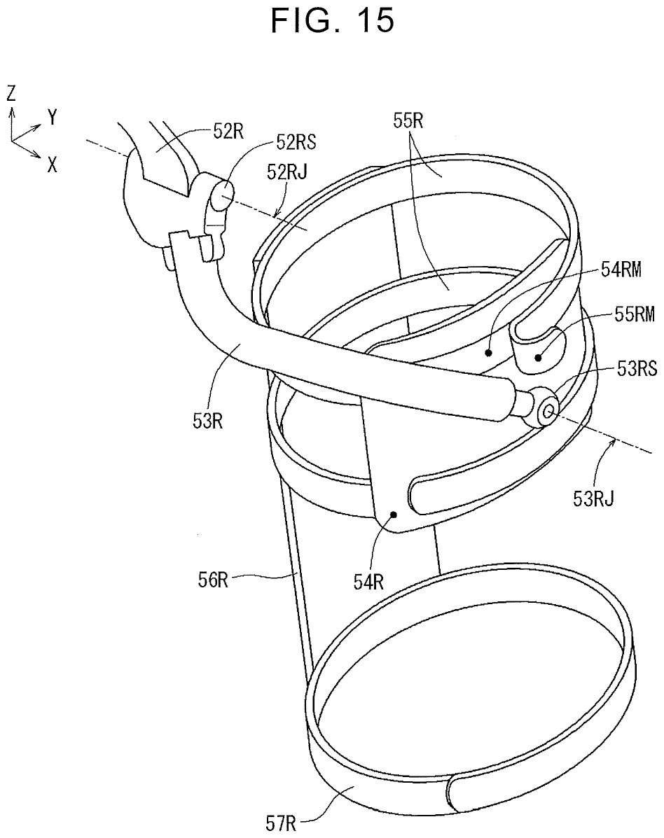

FIG. 14 shows an example in which the body holding portion is made up of the thigh worn portion 54R and the thigh belt 55R. FIG. 15 shows an example in which the body holding portion is made up of the thigh worn portion 54R, the thigh belt 55R, and a below-knee belt 57R. As shown in FIG. 15, the thigh belt 55R is provided at the thigh worn portion 54R so as to wrap the thigh above the knee of the user. The below-knee belt 57R is provided so as to wrap the below-knee region of the user. The below-knee belt 57R is made of the same material as the thigh belt 55R. As well as the thigh belt 55R, the below-knee belt 57R has a hook-and-loop fastener and is brought into close contact with the below-knee region. The thigh belt 55R and the below-knee belt 57R are coupled by a coupling member 56R on the back side of the knee of the user. The coupling member 56R extends from the thigh of the user toward the foot. The coupling member 56R is disposed behind the knee of the user. The coupling member 56R is made of a material that is able to bend in response to flexion or extension of the knee of the user. In this way, the thigh belt 55R is held in close contact with the above-knee region of the user, and the below-knee belt 57R is held in close contact with the below-knee region of the user.

The output link 50RA shown in FIG. 13 is made up of a plurality of coupling members in a manner such that the assist arm 51R (which may be regarded as the first link), a second link 52RA (and the second joint 52RS), a third link 53RA, and the thigh worn portion 54R (which may be regarded as the body holding portion) are coupled by joints. The thigh worn portion 54R shown in FIG. 13 does not show the thigh belt 55R shown in FIG. 14.

An end of the second link 52RA is coupled to the distal end of the assist arm 51R by the first joint 51RS so as to be pivotable about the pivot axis 51RJ. The first joint 51RS has a coupling structure with one degree of freedom such that the second link 52RA is pivotable about the pivot axis 51RJ relative to the assist arm 51R.

The second link 52RA and the second joint 52RS are integrated. One-end side of the third link 53RA is coupled to the second link 52RA by the second joint 52RS. The third link 53RA is reciprocally slidable along a slide axis 52RSJ that is the longitudinal direction of the third link 53RA. The second joint 52RS has a coupling structure with one degree of freedom such that the third link 53RA is slidable along the slide axis 52RSJ relative to the second link 52RA.

The third link 53RA is coupled to the thigh worn portion 54R by the third joint 53RS (spherical joint in the example of FIG. 13). Therefore, the third joint 53RS between the third link 53RA and the thigh worn portion 54R (body holding portion) has a coupling structure with three degrees of freedom. From above, the total number of degrees of freedom of the output link 50RA shown in FIG. 13 is 1+1+3=5.

Since the total number of degrees of freedom just needs to be greater than or equal to three, the third joint 53RS may have a coupling structure with one degree of freedom such that the thigh worn portion 54R is pivotable about the pivot axis 53RJ, as shown in FIG. 14. A stopper that limits the pivotable range of the second link 52RA or the slidable range of the third link 53RA may be provided.

FIG. 16 is a view illustrating an example in which the third joint 53RS that is the coupling portion between the third link 53RA and the thigh worn portion 54R in the link mechanism shown in FIG. 12 is disposed on the front of the thigh of the user. FIG. 17 is a view illustrating an example in which the third joint 53RS that is the coupling portion between the third link 53RA and the thigh worn portion 54R in the link mechanism shown in FIG. 12 is disposed on the side that is the outer side of the thigh of the user. FIG. 18 is a view illustrating an example in which the third joint 53RS that is the coupling portion between the third link 53RA and the thigh worn portion 54R in the link mechanism shown in FIG. 12 is disposed on the back of the thigh of the user. The location of the third joint 53RS may be any one of the front, side, and back of the thigh of the user.

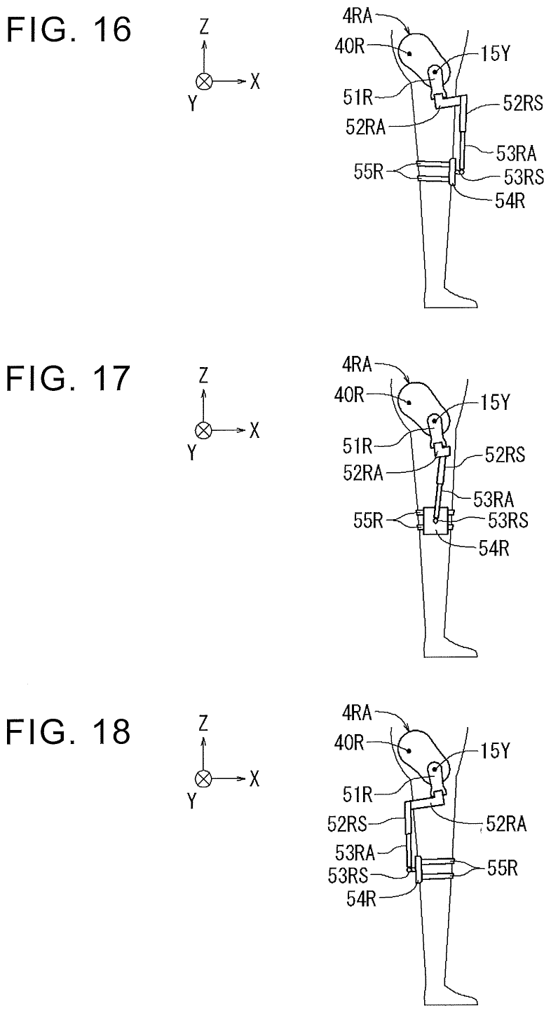

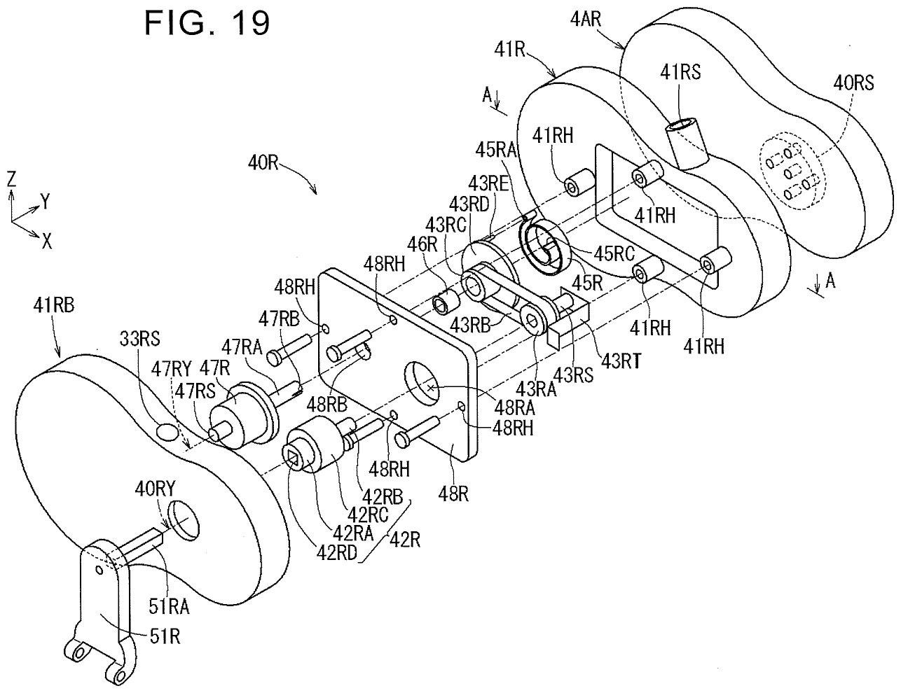

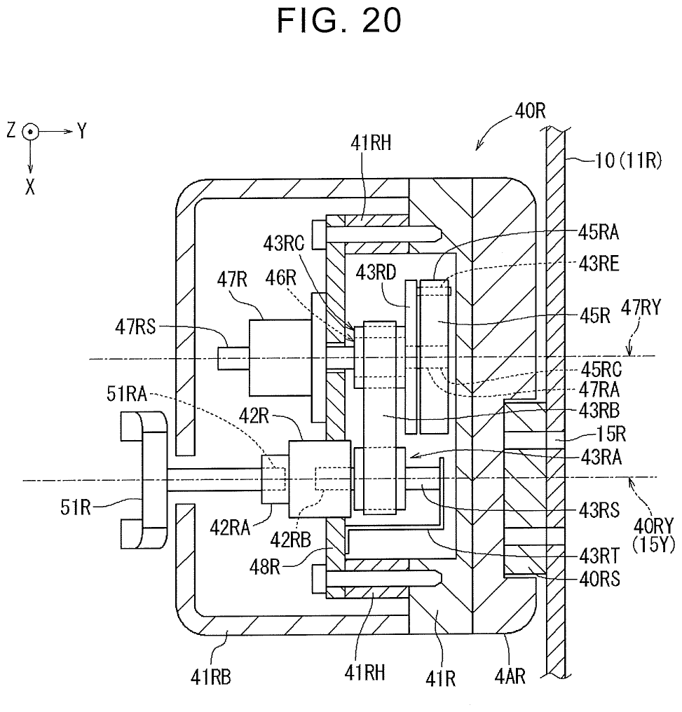

Next, parts accommodated in the cover 41RB of the torque generation unit 40R (see FIG. 5) will be described with reference to FIG. 19 and FIG. 20. FIG. 20 is a cross-sectional view taken along the line A-A in FIG. 19. As shown in FIG. 19 and FIG. 20, a speed reduction gear 42R, a pulley 43RA, a transmission belt 43RB, a pulley 43RC with a flange 43RD, a spiral spring 45R, a bearing 46R, an electric motor 47R (actuator), a sub-frame 48R, and other parts are accommodated in the cover 41RB. The assist arm 51R having a shaft 51RA is disposed on the outer side of the cover 41RB.

A cable drawing port 33RS (connection port) for drawing cables to drive, control, and communicate with the actuator is provided at a portion near the frame part 30 in the actuator unit 4R. A cable drawing port 33LS (connection port) for drawing cables to drive, control, and communicate with the actuator is provided at a portion near the frame part 30 in the actuator unit 4L. Cables (not shown) drawn out from the cable drawing ports 33RS, 33LS are disposed along the frame part 30 and connected to the backpack part 37.

As shown in FIG. 20, the torque generation unit 40R includes the actuator base part 41R, the cover 41RB, and the coupling base 4AR. The sub-frame 48R on which the electric motor 47R and other parts are mounted is connected to the actuator base part 41R. The cover 41RB is attached to one side of the actuator base part 41R. The coupling base 4AR is connected to the other side of the actuator base part 41R. The coupling base 4AR includes the coupling portion 40RS that is pivotable about the pivot axis 40RY.

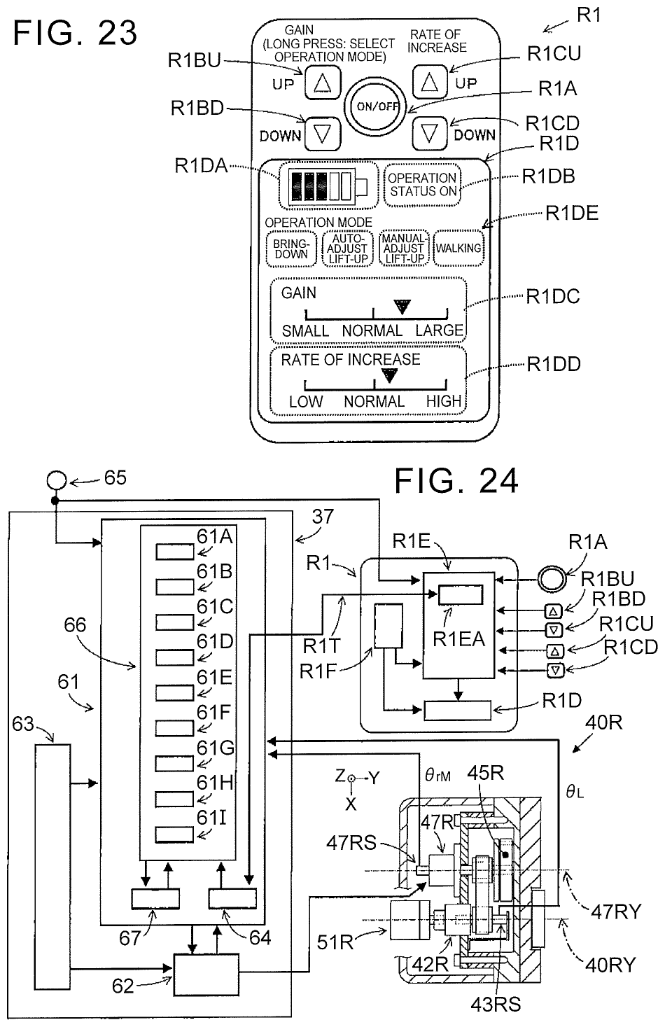

As shown in FIG. 19 and FIG. 20, an output link pivot angle detection unit 43RS (such as a pivot angle sensor) is connected to the pulley 43RA connected to a speed increasing shaft 42RB of the speed reduction gear 42R. The output link pivot angle detection unit 43RS detects a pivot angle of the assist arm 51R relative to the actuator base part 41R. The output link pivot angle detection unit 43RS is, for example, an encoder or an angle sensor. The output link pivot angle detection unit 43RS outputs a detected signal commensurate with a pivot angle to the controller 61 (see FIG. 24). A motor rotation angle detection unit 47RS is provided in the electric motor 47R. The motor rotation angle detection unit 47RS is able to detect a rotation angle of a motor shaft (which may be regarded as an output shaft). The motor rotation angle detection unit 47RS is, for example, an encoder or an angle sensor. The motor rotation angle detection unit 47RS outputs a detected signal commensurate with a rotation angle to the controller 61 (see FIG. 24).

As shown in FIG. 19, the sub-frame 48R has a through-hole 48RA and a through-hole 48RB. The through-hole 48RA holds a speed reduction gear housing 42RC of the speed reduction gear 42R. The through-hole 48RB allows an output shaft 47RA of the electric motor 47R to pass therethrough. The shaft 51RA of the assist arm 51R is fitted into a hole 42RD of a speed reduction shaft 42RA of the speed reduction gear 42R. The speed reduction gear housing 42RC of the speed reduction gear 42R is fixed to the sub-frame 48R in the through-hole 48RA. Thus, the assist arm 51R is supported so as to be pivotable about the pivot axis 40RY relative to the actuator base part 41R, and pivots integrally with the speed reduction shaft 42RA. The electric motor 47R is fixed to the sub-frame 48R. The output shaft 47RA is inserted through the through-hole 48RB of the sub-frame 48R. The sub-frame 48R is fixed to mounting portions 41RH of the actuator base part 41R by fastening members, such as bolts.

As shown in FIG. 19, the pulley 43RA is connected to the speed increasing shaft 42RB of the speed reduction gear 42R, and the output link pivot angle detection unit 43RS is connected to the pulley 43RA. A support member 43RT fixed to the sub-frame 48R is connected to the output link pivot angle detection unit 43RS. Thus, the output link pivot angle detection unit 43RS is able to detect a pivot angle of the speed increasing shaft 42RB relative to the sub-frame 48R (that is, relative to the actuator base part 41R). In addition, the pivot angle of the assist arm 51R is a pivot angle increased by the speed increasing shaft 42RB of the speed reduction gear 42R, so the output link pivot angle detection unit 43RS and the controller are able to detect a pivot angle of the assist arm 51R with a higher degree of resolution. Since the output link pivot angle detection unit 43RS and the controller detect a pivot angle of the output link with a higher degree of resolution, the controller is able to execute control with a higher precision. The shaft 51RA of the assist arm 51R, the speed reduction gear 42R, the pulley 43RA, and the output link pivot angle detection unit 43RS are disposed coaxially along the pivot axis 40RY.

The speed reduction gear 42R has a set reduction gear ratio n (1<n). When the speed reduction shaft 42RA is pivoted by a pivot angle θ, the speed reduction gear 42R pivots the speed increasing shaft 42RB by the pivot angle nθ. When the speed increasing shaft 42RB is pivoted by a pivot angle nθ, the speed reduction gear 42R pivots the speed reduction shaft 42RA by the pivot angle θ. The transmission belt 43RB is wound around the pulley 43RC and the pulley 43RA to which the speed increasing shaft 42RB of the speed reduction gear 42R is connected. Therefore, user torque from the assist arm 51R is transmitted to the pulley 43RC via the speed increasing shaft 42RB, and assist torque from the electric motor 47R is transmitted to the speed increasing shaft 42RB via the spiral spring 45R and the pulley 43RC.

The spiral spring 45R has a spring constant Ks. The spiral spring 45R has a spiral shape and has an inner end portion 45RC at the center side and an outer end portion 45RA at the outer peripheral side. The inner end portion 45RC of the spiral spring 45R is fitted into a groove 47RB formed in the output shaft 47RA of the electric motor 47R. The outer end portion 45RA of the spiral spring 45R is rolled in a cylindrical shape. A transmission shaft 43RE provided on the flange 43RD of the pulley 43RC is fitted to the outer end portion 45RA, so the outer end portion 45RA is supported by the transmission shaft 43RE (in the pulley 43RC, the flange 43RD and the transmission shaft 43RE are integrated). The pulley 43RC is supported so as to be pivotable about a pivot axis 47RY. The pulley 43RC has the transmission shaft 43RC near the outer periphery of the flange 43RD integrated with the pulley 43RC. The transmission shaft 43RE protrudes toward the spiral spring 45R. The transmission shaft 43RE is fitted into the outer end portion 45RA of the spiral spring 45R. The transmission shaft 43RE moves the location of the outer end portion 45RA around the pivot axis 47RY. The bearing 46R is provided between the output shaft 47RA of the electric motor 47R and the pulley 43RC. That is, the output shaft 47RA is not fixed to the pulley 43RC, and the output shaft 47RA is freely rotatable relative to the pulley 43RC. The pulley 43RC is driven for rotation by the electric motor 47R via the spiral spring 45R. With the above configuration, the output shaft 47RA of the electric motor 47R, the bearing 46R, the pulley 43RC with the flange 43RD, and the spiral spring 45R are disposed coaxially along the pivot axis 47RY.

The spiral spring 45R stores assist torque transmitted from the electric motor 47R and also stores user torque transmitted as a result of movement of the thigh of the user via the assist arm 51R, the speed reduction gear 42R, the pulley 43RA, and the pulley 43RC. As a result, the spiral spring 45R stores the resultant torque of the assist torque and the user torque. The resultant torque stored in the spiral spring 45R pivots the assist arm 51R via the pulley 43RC, the pulley 43RA, and the speed reduction gear 42R. With the above configuration, the output shaft 47RA of the electric motor 47R is connected to the output link (the assist arm 51R in the case of FIG. 19) via the speed reduction gear 42R. The speed reduction gear 42R reduces the rotation angle of the output shaft 47RA.

The resultant torque stored in the spiral spring 45R is found based on the amount of change in angle from a no-load state and the spring constant. The resultant torque is found based on, for example, the pivot angle of the assist arm 51R (acquired by the output link pivot angle detection unit 43RS), the rotation angle of the output shaft 47RA of the electric motor 47R (acquired by the motor rotation angle detection unit 47RS), and the spring constant Ks of the spiral spring 45R. The user torque is extracted from the found resultant torque, and assist torque commensurate with the user torque is output from the electric motor.

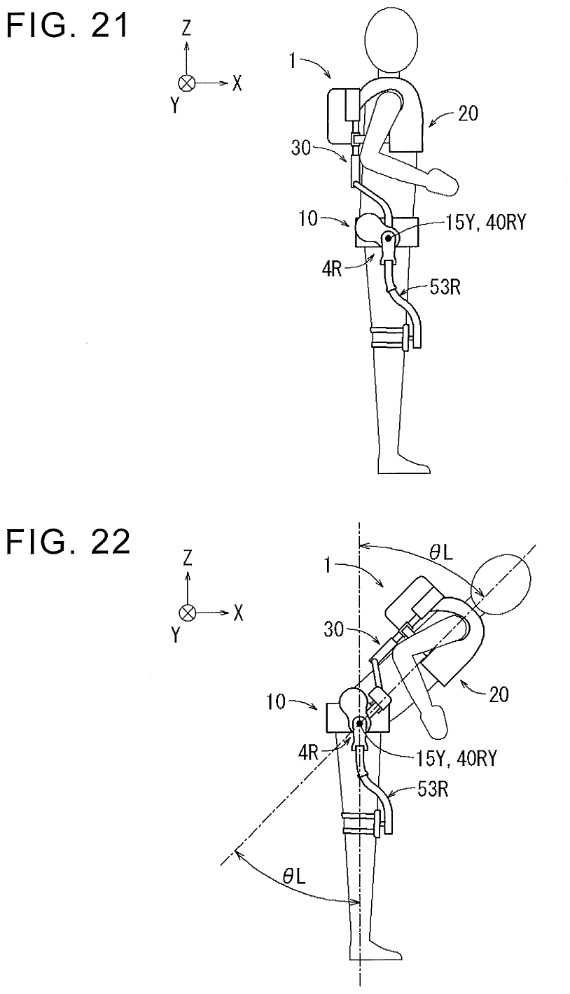

As shown in FIG. 20, the torque generation unit 40R of the right actuator unit 4R includes the coupling portion 40RS pivotable about the pivot axis 40RY (that is, the assumed pivot axis 15Y). As shown in FIG. 3 and FIG. 1, the coupling portion 40RS is coupled (fixed) by coupling members, such as bolts, via the mounting holes 15R of the lower back support part 10. As shown in FIG. 3 and FIG. 1, the lower end of the right sub-frame 32R of the frame part 30 is connected (fixed) to the connection portion 41RS of the right actuator unit 4R. Similarly, the coupling portion 40LS of the torque generation unit 40L of the left actuator unit 4L is coupled (fixed) to coupling members, such as bolts, via the mounting holes 15L of the lower back support part 10, and the lower end of the left sub-frame 32L of the frame part 30 is connected (fixed) to the connection portion 41LS of the left actuator unit 4L. That is, as shown in FIG. 3, the lower back support part 10 and the frame part 30 are fixed to the torque generation unit 40R of the right actuator unit 4R, and the lower back support part 10 and the frame part 30 are fixed to the torque generation unit 40L of the left actuator unit 4L. The right actuator unit 4R, the left actuator unit 4L, and the frame part 30 are integrated. The right actuator unit 4R is pivotable relative to the lower back support part 10 at the coupling portion 40RS pivotable about the assumed pivot axis 15Y (see FIG. 21 and FIG. 22). The left actuator unit 4L is pivotable relative to the lower back support part 10 at the coupling portion 40LS pivotable about the assumed pivot axis 15Y (see FIG. 21 and FIG. 22).

Next, the operating unit R1 for the user to easily adjust the assist status of the assist device 1 or perform other operations will be described with reference to FIG. 23 to FIG. 25. As shown in FIG. 24, the operating unit R1 is connected to the controller 61 in the backpack part 37 (see FIG. 1) by a wired or wireless communication line R1T. A controller R1E of the operating unit R1 is able to exchange information with the controller 61 via a communication unit R1EA. The controller 61 is able to exchange information with the controller R1E in the operating unit R1 via a communication unit 64. As shown in FIG. 1, when the user does not operate the operating unit R1, the user can, for example, put the operating unit R1 in the holder R1S, such as a pocket, provided on the jacket part 20 (see FIG. 1).

As shown in FIG. 23, the operating unit R1 includes a main control button R1A, a gain UP control button R1BU, a gain DOWN control button R1BD, a rate-of-increase UP control button R1CU, a rate-of-increase DOWN control button R1CD, a display RID, and other components. The gain UP control button R1BU and the gain DOWN control button R1BD may be regarded as a gain change unit. The rate-of-increase UP control button R1CU and the rate-of-increase DOWN control button R1CD may be regarded as a rate-of-increase change unit. As shown in FIG. 24, the controller R1E, a power supply R1F for the operating unit, and other components are provided in the operating unit R1. The main control button R1A, the gain UP control button R1BU, the gain DOWN control button R1BD, the rate-of-increase UP control button R1CU, and the rate-of-increase DOWN control button R1CD may be configured not to project from the surface on which these components are disposed, to prevent misoperation while the operating unit R1 is put in the holder R1S (see FIG. 1).

The main control button R1A is a switch to cause the assist device 1 to start or stop assist control when operated by the user. As shown in FIG. 24, a main power switch 65 to cause the assist device 1 itself (as a whole) to start up or shut down is provided at, for example, the backpack part 37. When the main power switch 65 is operated into ON position, the controller 61 and the controller R1E start up. When the main power switch 65 is operated into OFF position, the controller 61 and the controller R1E shut down. As shown in FIG. 23, a display area R1DB on the display RID of the operating unit R1, for example, shows whether the current operational status of the assist device 1 is ON (operating) or OFF (stopped).

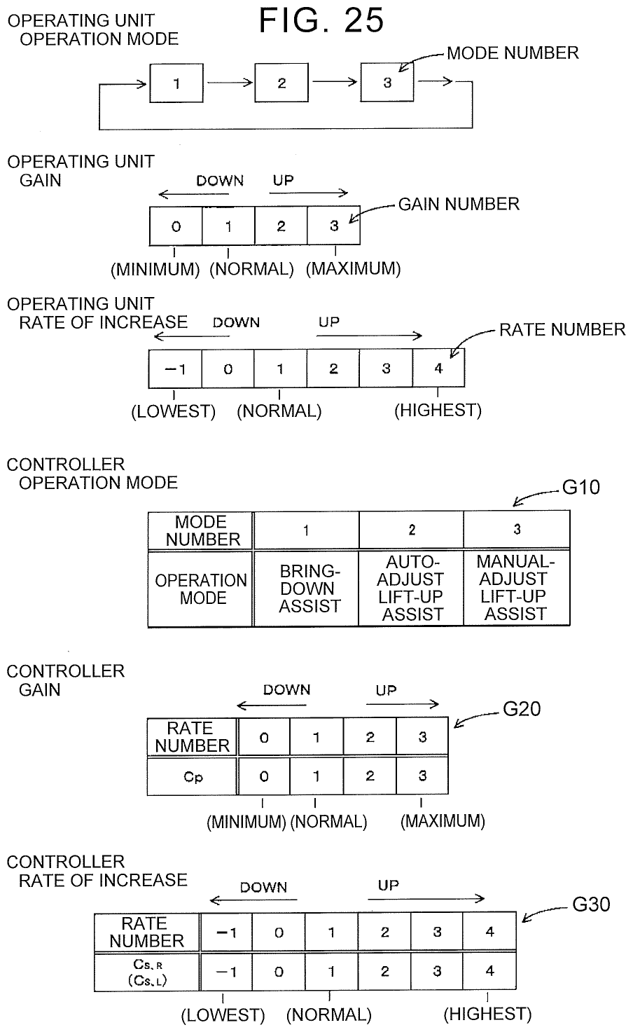

The gain UP control button R1BU is a switch to increase the gain of assist torque that the assist device 1 generates when operated by the user. The gain DOWN control button R1BD is a switch to reduce the gain of assist torque that the assist device 1 generates when operated by the user. For example, as shown in “OPERATING UNIT GAIN” of FIG. 25, the controller R1E increments the stored gain number by one each time the gain UP control button R1BU is operated, and decrements the gain number by one each time the gain DOWN control button R1BD is operated. In the example of FIG. 25, an example in which the number of gain numbers is four, that is, 0 to 3, is shown; however, the number of gain numbers is not limited to four. As shown in FIG. 24, the controller R1E, for example, shows display for the current gain number in the display area R1DC on the display RID of the operating unit R1.

When the gain UP control button R1BU is pressed and held for, for example, five seconds or longer, the gain UP control button R1BU functions as an operation mode select switch. When the gain UP control button R1BU is pressed and held, the operation mode (mode number) shifts in turn in order of “1 (bring-down assist mode)”, “2 (auto-adjust lift-up assist mode)”, and “3 (manual-adjust lift-up assist mode)” as shown in “OPERATING UNIT OPERATION MODE” of FIG. 25 each time the gain UP. control button R1BU is pressed. In this case, the gain UP control button R1BU may be regarded as an operation mode select unit. As shown in FIG. 23, the controller R1E, for example, shows display for the current operation mode in the display area R1DB on the display RID of the operating unit R1. Walking mode cannot be selected by the gain UP control button R1BU; however, when the controller 61 recognizes that the user is walking, the operation mode automatically shifts into the walking mode.

The rate-of-increase UP control button R1CU is a switch to increase the rate of increase in assist torque that the assist device 1 generates when operated by the user. The rate-of-increase DOWN control button R1CD is a switch to decrease the rate of increase in assist torque that the assist device 1 generates when operated by the user. For example, as shown in “OPERATING UNIT RATE OF INCREASE” of FIG. 25, the controller R1E increments the stored rate number by one each time the rate-of-increase UP control button R1CU is operated, and decrements the rate number by one each time the rate-of-increase DOWN control button R1CD is operated. In the example of FIG. 25, an example in which six rate numbers, that is, −1 to 4, are shown; however, the number of rate numbers is not limited to six. As shown in FIG. 24, the controller R1E, for example, shows display for the current rate number in a display area R1DD on the display R1D of the operating unit R1.

The controller R1E of the operating unit R1 transmits operation information via the communication unit R1EA (see FIG. 24) at predetermined time intervals (for example, intervals of several milliseconds to several hundreds of milliseconds) or each time any one of the main control button R1A, the gain UP control button R1BU, the gain DOWN control button R1BD, the rate-of-increase UP control button R1CU, and the rate-of-increase DOWN control button R1CD is operated. The operation information contains the above-described shutdown instruction or startup instruction, mode number, gain number, rate number, or other information.

As the controller 61 of the backpack part 37 receives operation information, the controller 61 stores the received operation information, and transmits response information via the communication unit 64 (see FIG. 24). The response information contains battery information indicating the status of battery of the power supply unit 63 that is used to drive the assist device 1, assist information indicating the assist status, or other information. The battery information contained in the response information contains the level of the power supply unit 63 or other information. The assist information contained in the response information contains, for example, error information indicating the details of malfunction when a malfunction is found from the assist device 1. As shown in FIG. 24, the controller R1E, for example, shows battery level or other information in a display area R1DA on the display R1D of the operating unit R1, and, when the response information contains error information, shows error information at any location on the display RID.

As the controller 61 (see FIG. 24) receives operation information from the controller R1E, the controller 61 starts up the assist device 1 when the received operation information contains the startup instruction, and shuts down the assist device 1 when the received operation information contains the shutdown instruction. For example, as shown in “CONTROLLER OPERATION MODE” of FIG. 25, the controller 61 stores operation mode in association with the received mode number. For example, as shown in “CONTROLLER GAIN” of FIG. 25, the controller 61 stores a gain Cₚ (0 to 3) in association with the gain number, and stores a rate of increase (right) C_{s,R} (rate number for right: −1 to 4) and a rate of increase (left) C_{s,L} (rate number for left: −1 to 4) in association with the rate number. The operation mode, Cₚ, C_{s,R}, and C_{s,L} will be used in procedures described later.

As described above, the user can easily perform adjustment for a desired assist status by operating the operating unit R1. Since battery level, error information, or other information is displayed on the display RID of the operating unit R1, the user can easily get the status of the assist device 1. Forms of various pieces of information that are displayed on the display RID are not limited to the example of FIG. 23.

As shown in FIG. 24, the controller 61 is accommodated in the backpack part 37. In the example shown in FIG. 24, the controller 61, a motor driver 62, the power supply unit 63, and other components are accommodated in the backpack part 37. The controller 61 includes, for example, a control unit 66 (CPU) and a storage unit 67 (which stores control programs or other information). The controller 61 includes an adjustment determination unit 61A, an input processing unit 61B, a torque change amount calculation unit 61C, a motion type determination unit 61D, a selection unit 61E, a bring-down assist torque calculation unit 61F, a lift-up assist torque calculation unit 61G, a walking assist torque calculation unit 61H, a control command value calculation unit 61I, the communication unit 64, and other components (described later). The motor driver 62 is an electronic circuit that outputs a drive current to drive the electric motor 47R based on a control signal from the controller 61. The power supply unit 63 is, for example, a lithium battery. The power supply unit 63 supplies electric power to the controller 61 and the motor driver 62. The operation and other details of the communication unit 64 will be described later.

Operation information from the operating unit R1, a detected signal (detected signal commensurate with an actual motor shaft angle θ_{rM} of the electric motor 47R) from the motor rotation angle detection unit 47RS, a detected signal (detected signal commensurate with an actual link angle θ_{L} of the assist arm 51R) from the output link pivot angle detection unit 43RS, and other information are input to the controller 61. The controller 61 finds a rotation angle of the electric motor 47R based on input signals, and outputs a control signal commensurate with the found rotation angle to the motor driver 62.

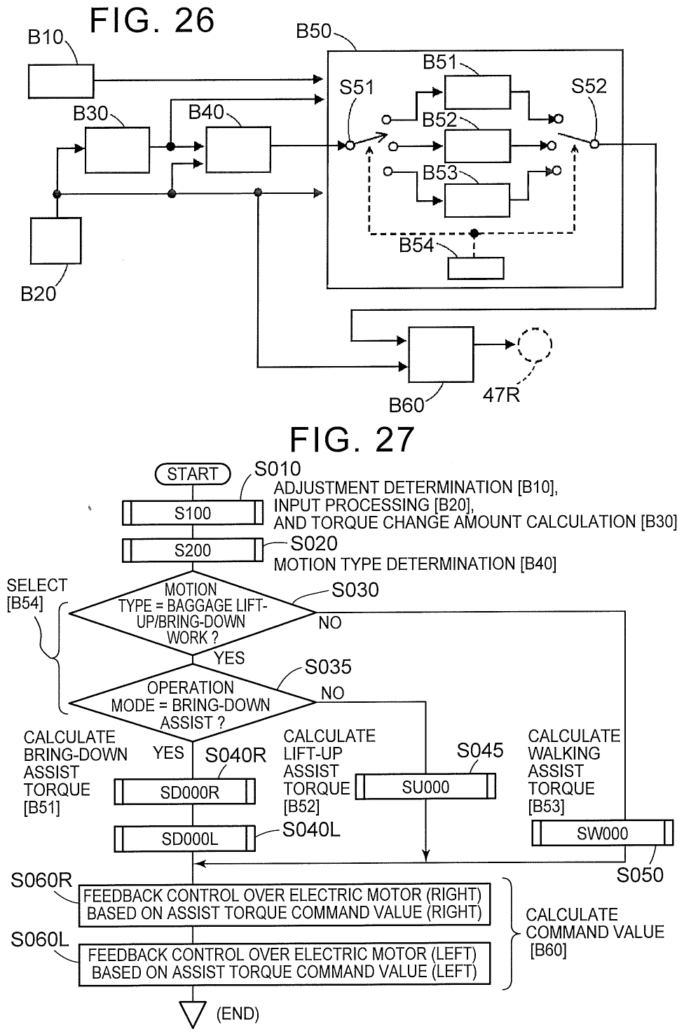

Next, the procedure of the controller 61 will be described with reference to the flowchart shown in FIG. 27 and control blocks shown in FIG. 26. The control blocks shown in FIG. 26 include an adjustment determination block B10, an input processing block B20, a torque change amount calculation block B30, a motion type determination block B40, a selection block B54, a bring-down assist torque calculation block B51, a lift-up assist torque calculation block B52, a walking assist torque calculation block B53, a control command value calculation block B60, select switches S51, S52, and the like. The details of processes of the blocks will be described with reference to the flowchart shown in FIG. 27.

The flowchart shown in FIG. 27 shows a procedure to control the right actuator unit 4R and the left actuator unit 4L. The process shown in FIG. 27 is started at predetermined time intervals (for example, intervals of several milliseconds). As the process is started, the controller 61 advances the process to step S010.

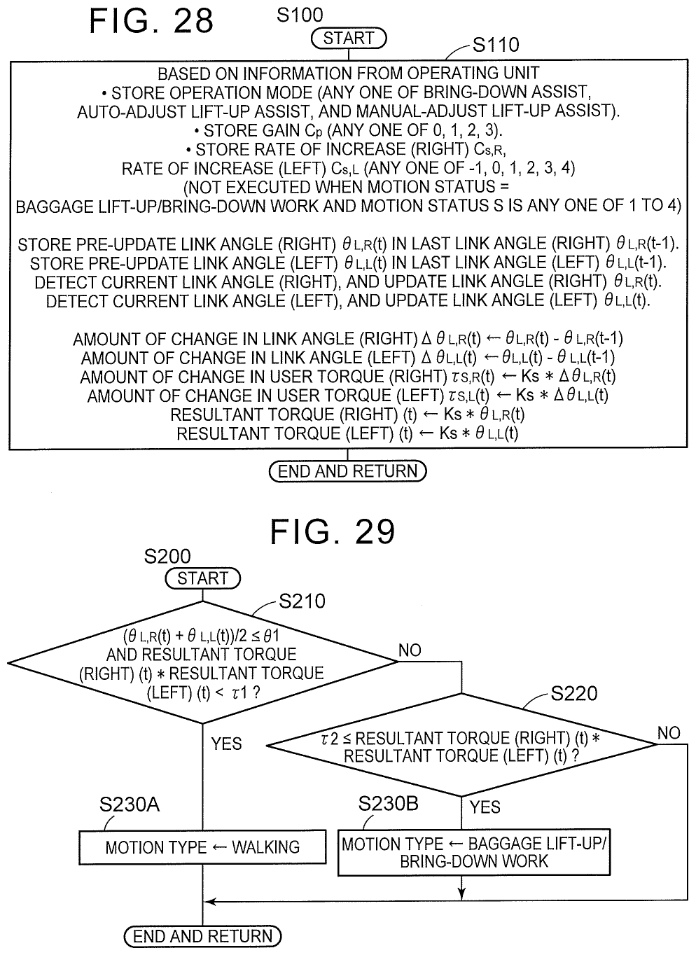

In step S010, the controller 61 executes the process of S100 (see FIG. 28), and then advances the process to step S020. The process of S100 may be regarded as the adjustment determination block B10, the input processing block B20, and the torque change amount calculation block B30 shown in FIG. 26, and may be regarded as the adjustment determination unit 61A, the input processing unit 61B, and the torque change amount calculation unit 61C shown in FIG. 24. The details of the process of S100 will be described later.

In step S020, the controller 61 executes the process of S200 (see FIG. 29), and then advances the process to step S030. The process of S200 may be regarded as the motion type determination block B40 shown in FIG. 26, and may be regarded as the motion type determination unit 61D shown in FIG. 24. The details of the process of S200 will be described later.

In step S030, the controller 61 determines whether the motion type determined in step S020 is baggage lift-up/bring-down work. When the motion type is the baggage lift-up/bring-down work (Yes), the controller 61 advances the process to step S035; otherwise (No), the controller 61 advances the process to step S050. A control block B50 shown in FIG. 26 is a control block for determining and selecting lift-up assist mode, bring-down assist more or walking assist mode.

When the controller 61 has advanced the process to step S035, the controller 61 determines whether the operation mode (operation mode from the operating unit) acquired in step S010 is bring-down assist mode. When the operation mode is bring-down assist mode (Yes), the controller 61 advances the process to step S040R; otherwise (No), the controller 61 advances the process to step S045. The processes of step S030 and step S035 may be regarded as the selection block B54 shown in FIG. 26, and may be regarded as the selection unit 61E shown in FIG. 24.

When the controller 61 has advanced the process to step S040R, the controller 61 executes the process of SD000R (see FIG. 30), and then advances the process to step S040L. The process of SD000R is a process of finding a control command value for the right actuator unit 4R during bring-down motion. This process may be regarded as the bring-down assist torque calculation block B51 shown in FIG. 26, and may be regarded as the bring-down assist torque calculation unit 61F in FIG. 24. The details of the process of SD000R will be described later.

In step S040L, the controller 61 executes the process of SD000L (not shown), and then advances the process to step S060R. The process of SD000L is a process of finding a control command value for the left actuator unit 4L during bring-down motion. This process may be regarded as the bring-down assist torque calculation block B51 shown in FIG. 26, and may be regarded as the bring-down assist torque calculation unit 61F shown in FIG. 24. The process of SD000L is similar to that of SD000R, so the detailed description thereof is omitted.

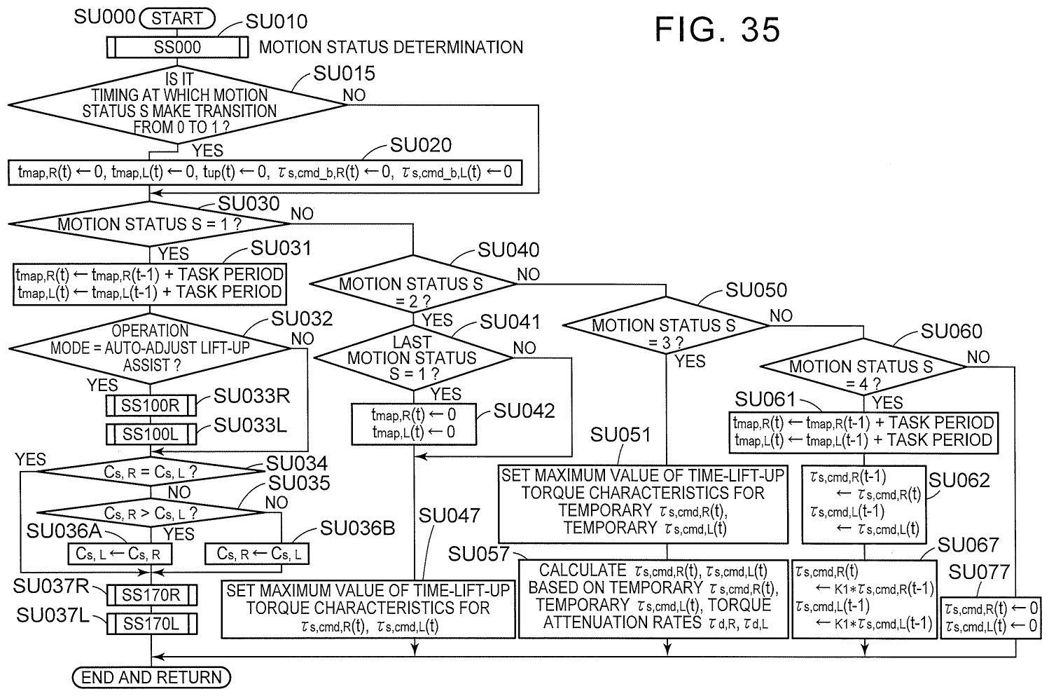

When the controller 61 has advanced the process to step S045, the controller 61 executes the process of SU000 (see FIG. 35), and then advances the process to step S060R. The process of SU000 is a process of finding control command values for the right actuator unit 4R and the left actuator unit 4L during lift-up motion. This process may be regarded as the lift-up assist torque calculation block B52 shown in FIG. 26, and may be regarded as the lift-up assist torque calculation unit 61G shown in FIG. 24. The details of the process of SU000 will be described later.

When the controller 61 has advanced the process to step S050, the controller 61 executes the process of SW000 (not shown), and then advances the process to step S060R. The process of SW000 is a process of finding control command values for the right actuator unit 4R and the left actuator unit 4L during walking motion. This process may be regarded as the walking assist torque calculation block B53 shown in FIG. 26, and may be regarded as the walking assist torque calculation unit 61H shown in FIG. 24. The detailed description of the process of SW000 is omitted.

In step S060R, the controller 61 executes feedback control over the electric motor (right) based on the assist torque command value (right) found in SD000R or SU000 or SW000, and then advances the process to step S060L.

In step S060L, the controller 61 executes feedback control over the electric motor (left) based on the assist torque command value (left) found in SD000L or SU000 or SW000, and then ends the process. The processes of step S060R and step S060L may be regarded as the control command value calculation block B60 shown in FIG. 26, and may be regarded as the control command value calculation unit 61I shown in FIG. 24.

Next, the details of the process of S100 that is step S010 shown in FIG. 27 will be described with reference to FIG. 28. In the process of S100, the controller 61 stores any one of bring-down assist mode, auto-adjust lift-up assist mode, and manual-adjust lift-up assist mode for the operation mode based on information from the operating unit (see “CONTROLLER OPERATION MODE” of FIG. 25). The controller 61 stores any one of 0, 1, 2, and 3 in the gain Cₚ based on the information from the operating unit (see “CONTROLLER GAIN” of FIG. 25). Except “the case the motion type is baggage lift-up/bring-down work and a motion status S is any one of 1 to 4, the controller 61 stores any one of −1, 0, 1, 2, 3, 4 in the rate of increase (right) C_{s,R} and the rate of increase (left) C_{s,L} based on the information from the operating unit (see “CONTROLLER RATE OF INCREASE” of FIG. 25). The above-described process may be regarded as the adjustment determination block B10 shown in FIG. 26, and may be regarded as the adjustment determination unit 61A shown in FIG. 24.

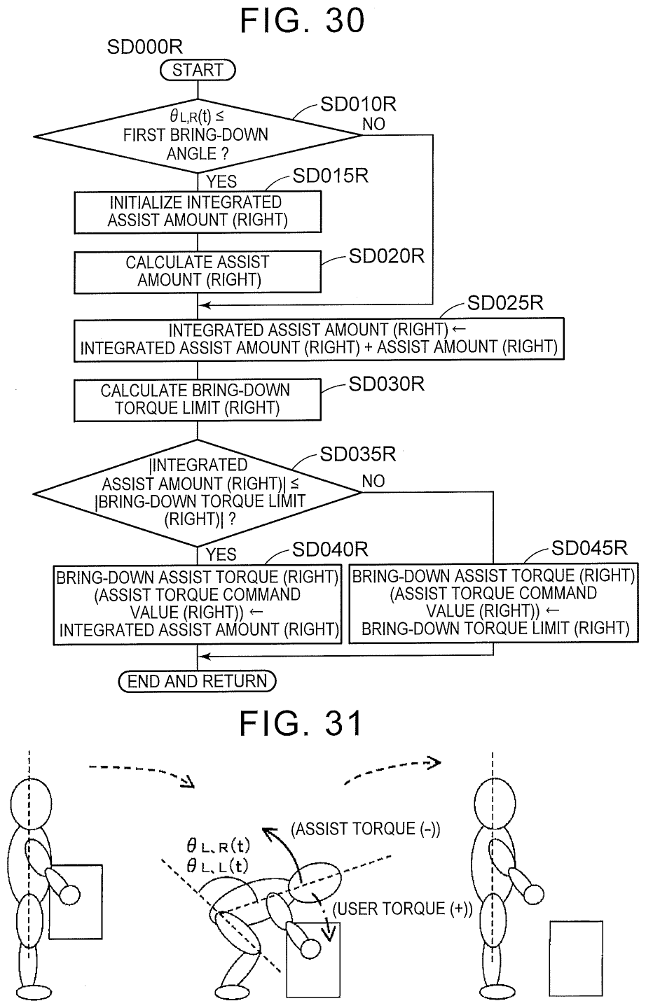

The controller 61 stores a pre-update link angle (right) θ_{L,R}(t) in a last link angle (right) θ_{L,R}(t−1), and stores a pre-update link angle (left) θ_{L,L}(t) in a last link angle (left) θ_{L,L}(t−1). The controller 61 detects a current link angle (right) with the use of the output link pivot angle detection unit 43RS for the right actuator unit 4R (which may be regarded as an angle detection unit, and see FIG. 19 and FIG. 20), and stores (updates) the link angle (right) θ_{L,R}(t). Similarly, the controller 61 detects a current link angle (left) with the use of the output link pivot angle detection unit for the left actuator unit 4L (which may be regarded as the angle detection unit), and stores (updates) the link angle (left) θ_{L,L}(t). The above-described process may be regarded as the input processing block B20 shown in FIG. 26, and may be regarded as the input processing unit 61B shown in FIG. 24. The link angle (right) θ_{L,R}(t) is a forward lean angle (right) of the lower back relative to the thigh (see FIG. 31). The link angle (left) θ_{L,L}(t) is a forward lean angle (left) of the lower back relative to the thigh (see FIG. 31).

The controller 61 finds an amount of change in link angle (right) Δθ_{L,R}(t) by using the following mathematical expression (1) and stores the amount of change in link angle (right) Δθ_{L,R}(t). The controller 61 finds an amount of change in link angle (left) Δθ_{L,L}(t) by using the following mathematical expression (2) and stores the amount of change in link angle (left) Δθ_{L,L}(t). The amount of change in link angle (right) Δθ_{L,R}(t) and the amount of change in link angle (left) Δθ_{L,L}(t) may be regarded as an angular velocity-related amount. The output link pivot angle detection unit 43RS may be regarded as a torque detection unit.

Amount of change in link angle(right)Δθ_{L,R}(t)=Link angle(right)θ_{L,R}(t)−Link angle (right)θ_{L,R}(t−1) (1)

Amount of change in link angle(left)Δθ_{L,L}(t)=Link angle(left)θ_{L,L}(t)−Link angle (left)θ_{L,L}(t−1) (2)

The controller 61 finds an amount of change in user torque (right) τ_{S,R}(t) by using the following mathematical expression (3) and stores the amount of change in user torque (right) τ_{S,R}(t). The controller 61 finds an amount of change in user torque (left) τ_{S,L}(t) by using the following mathematical expression (4) and stores the amount of change in user torque (left) τ_{S,L}(t). Ks is the spring constant of the spiral spring 45R.

Amount of change in user torque(right)τ_{S,R}(t)=Ks·Δθ_{L,R}(t) (3)

Amount of change in user torque(left)τ_{S,L}(t)=Ks·Δθ_{L,L}(t) (4)

The controller 61 finds a resultant torque (right) (t) by using the following mathematical expression (5) and stores the resultant torque (right) (t). The controller 61 finds a resultant torque (left) (t) by using the following mathematical expression (6) and stores the resultant torque (left) (t). The above-described process may be regarded as the torque change amount calculation block B30 shown in FIG. 26, and may be regarded as the torque change amount calculation unit 61C shown in FIG. 24.

Resultant torque(right)(t)=Ks·θ_{L,R}(t) (5)

Resultant torque(left)(t)=Ks·θ_{L,L},(t) (6)

Next, the details of the process of S200 that is step S020 shown in FIG. 27 will be described with reference to FIG. 29. In S200, the controller 61 determines the motion type of the user. The motion type to be determined includes “walking” and “baggage lift-up/bring-down”. The “walking” is the walking motion of the user. The “baggage lift-up/bring-down” is the motion that the user lifts up a heavy load or the motion that the user brings down a held heavy load. The process of S200 may be regarded as the motion type determination block B40 shown in FIG. 26, and may be regarded as the motion type determination unit 61D shown in FIG. 24.

In the process of S200, the controller 61 advances the process to step S210. In step S210, the controller 61 determines whether [Link angle (right) θ_{L,R}(t)+Link angle (left) θ_{L,L}(t)]/2 is less than or equal to a first motion determination angle θ1 and Resultant torque (right) (t)·Resultant torque (left) (t) is less than a first motion determination torque τ1. When [Link angle (right) θ_{L,R}(t)+Link angle (left) θ_{L,L}(t)]/2 is less than or equal to the first motion determination angle θ1 and Resultant torque (right) (t)·Resultant torque (left) (t) is less than the first motion determination torque τ1 (Yes), the controller 61 advances the process to step S230A; otherwise (No), the controller 61 advances the process to step S220.

When the controller 61 has advanced the process to step S220, the controller 61 determines whether Resultant torque (right) (t). Resultant torque (left) (t) is greater than or equal to a second motion determination torque τ2. When Resultant torque (right) (t). Resultant torque (left) (t) is greater than or equal to the second motion determination torque τ2 (Yes), the controller 61 advances the process to step S230B; otherwise (No), the controller 61 ends the process of S200 and returns (the controller 61 advances the process to step S030 of FIG. 27).

When the controller 61 has advanced the process to step S230A, the controller 61 stores “walking” in the motion type, and then ends the process of S200 and returns (the controller 61 advances the process to step S030 of FIG. 27).

When the controller 61 has advanced the process to step S230B, the controller 61 stores “baggage lift-up/bring-down work” in the motion type, and then ends the process of S200 and returns (the controller 61 advances the process to step S030 of FIG. 27).

Next, the details of the process of SD000R that is step S040R shown in FIG. 27 will be described with reference to FIG. 30. In SD000R, the controller 61 calculates a bring-down assist torque (right) to be generated by the assist device 1 to assist the user at bring-down work. The process of SD000R shows a procedure to calculate a bring-down assist torque (right) to be generated by the right actuator unit 4R (see FIG. 1). A procedure of SD000L (see FIG. 27) to calculate a bring-down assist torque (left) to be generated by the left actuator unit 4L (see FIG. 1) is similar to the procedure of SD000R, so the description thereof is omitted. As shown in FIG. 31, at bring-down work that the user brings down held baggage, the link angle (right) θ_{L,R}(t) and the link angle (left) θ_{L,L}(t) each are a forward lean angle of the lower back relative to the thigh. The bring-down assist torque to assist the user at work in a bring-down direction (the direction of “USER TORQUE” in FIG. 31) is generated in a lift-up direction (the direction of “ASSIST TORQUE” in FIG. 31) relative to the user. In the following description, the sign of torque in the lift-up direction is − (negative), and the sign of torque in the bring-down direction is + (positive).

In the process of SD000R, the controller 61 advances the process to step SD010R. In step SD010R, the controller 61 determines whether the link angle (right) θ_{L,R}(t) is less than or equal to a first bring-down angle θd1. When the link angle (right) θ_{L,R}(t) is less than or equal to the first bring-down angle d1 (Yes), the controller 61 advances the process to step SD015R; otherwise (No), the controller 61 advances the process to step SD020R. For example, the first bring-down angle θd1 is a forward lean angle of about 10°. When θ_{L,R}(t)≤θd1, the controller 61 determines that bring-down motion is started or bring-down motion is finished.

When the controller 61 has advanced the process to step SD015R, the controller 61 initializes an integrated assist amount (right) (the controller. 61 resets the integrated assist amount to zero) and then advances the process to step SD020R.

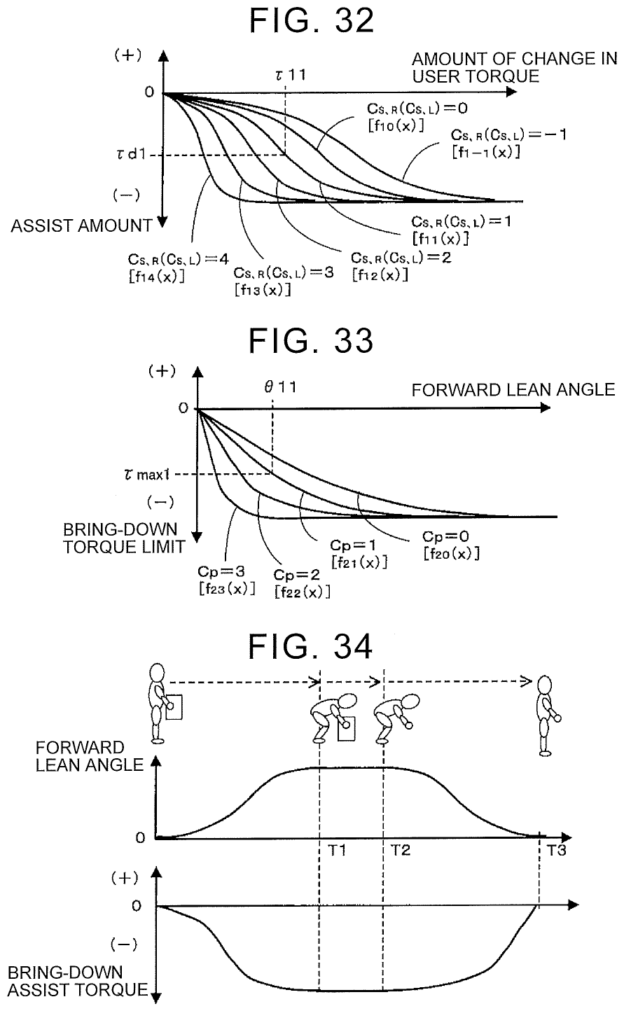

When the controller 61 has advanced the process to step SD020R, the controller 61 calculates an assist amount (right) based on the rate of increase (right) C_{s,R}, the amount of change in user torque (right) τ_{s,R}(t), and user torque change amount-assist amount characteristics (FIG. 32), and then advances the process to step SD025R. As shown in FIG. 32, for example, when Rate of increase (right) C_{s,R}=1 and Amount of change in user torque (right) τ_{S,R}(t)=τ11, τd1 for τ11 by using the characteristics of f11(x) for Cs=1 is the assist amount (right) to be found.

In step SD025R, the controller 61 adds the assist amount (right) found in step SD020R to the integrated assist amount (right) (that is, the controller 61 integrates the found assist amount (right)) and then advances the process to step SD030R.

In step SD030R, the controller 61 calculates a bring-down torque limit (right) based on the gain Cₚ, the link angle (right) (forward lean angle) θ_{L,R}(t), and forward lean angle-bring-down torque limit characteristics (see FIG. 33), and then advances the process to step SD035R. As shown in FIG. 33, for example, when the gain Cₚ=1 and Link angle (right) (forward lean angle) θ_{L,R}(t)=θ11, τmax1 for θ11 by using the characteristics of f21(x) for Cₚ=1 is the bring-down torque limit (right) to be found.

In step SD035R, the controller 61 determines whether |Integrated assist amount (right)| is less than or equal to |Bring-down torque limit (right)|. When |Integrated assist amount (right)| is less than or equal to |Bring-down torque limit (right)| (Yes), the controller 61 advances the process to step SD040R; otherwise (No), the controller 61 advances the process to step SD045R.

When the controller 61 has advanced the process to step SD040R, the controller 61 stores the integrated assist amount (right) in the bring-down assist torque (right) (that is, assist torque command value (right) τ_{s,cmd,R}(t)), and then ends the process and returns (the controller 61 advances the process to step S060R of FIG. 27).

When the controller 61 has advanced the process to step SD045R, the controller 61 stores the bring-down torque limit (right) in the bring-down assist torque (right) (that is, assist torque command value (right) τ_{s,cmd,R}(t)), and then ends the process and returns (the controller 61 advances the process to step S060R of FIG. 27).

In step SD035R, step SD040R, and step SD045R, the controller 61 sets a smaller one of |Integrated assist amount (right)| and |Bring-down torque limit (right)| for the bring-down assist torque (right).

FIG. 34 shows a scene where the bring-down assist torque is adapted to a forward lean angle at the time of bring-down work in the above-described process. The example shown in FIG. 34 shows the case the user holds baggage in an erect state at time 0, gradually increases the forward lean angle and completes bringing down the baggage at time T1, keeps the forward lean state until time T2, and gradually reduces the forward lean angle back to the erect state. In this case, the bring-down assist torque in the lift-up direction (toward − (negative) side in FIG. 34) is as shown in FIG. 34, which reduces a load on the lower back of the user and appropriately assists at bring-down work.

When the user stops forward bending motion and then a change in forward lean angle stops (Δθ_{L,R}(t)=0, Δ_{L,L}(t)=0) (in the example of FIG. 34, from time T1 to time T2) or during time of erect motion when the user gradually reduces the forward lean angle from a forward bending state (in the example of FIG. 34, from time T2 to time T3), the amount of change in user torque is zero or in an opposite direction, so the assist amount that is determined based on the user torque change amount-assist amount characteristics (see FIG. 32) is zero. That is, in this case, the controller 61 stops updating the integrated assist amount and holds the integrated assist amount, and finds a bring-down assist torque (right) (assist torque command value (right)) based on the held integrated assist amount and the bring-down torque limit.

Next, the details of the process of SU000 that is step S045 shown in FIG. 27 will be described with reference to FIG. 35. In SU000, the controller 61 calculates a lift-up assist torque to be generated by the assist device 1 to assist the user at lift-up work. At lift-up work that the user lifts up baggage, the link angle (right) θ_{L,R}(t) and the link angle (left) θ_{L,L}(t) (see FIG. 31) each are a forward lean angle of the lower back relative to the thigh. The lift-up assist torque to assist the user at work in the lift-up direction is generated in the lift-up direction (the direction of “ASSIST TORQUE” in FIG. 31) relative to the user. In the following description, the sign of torque in the lift-up direction is − (negative), and the sign of torque in the bring-down direction is + (positive).

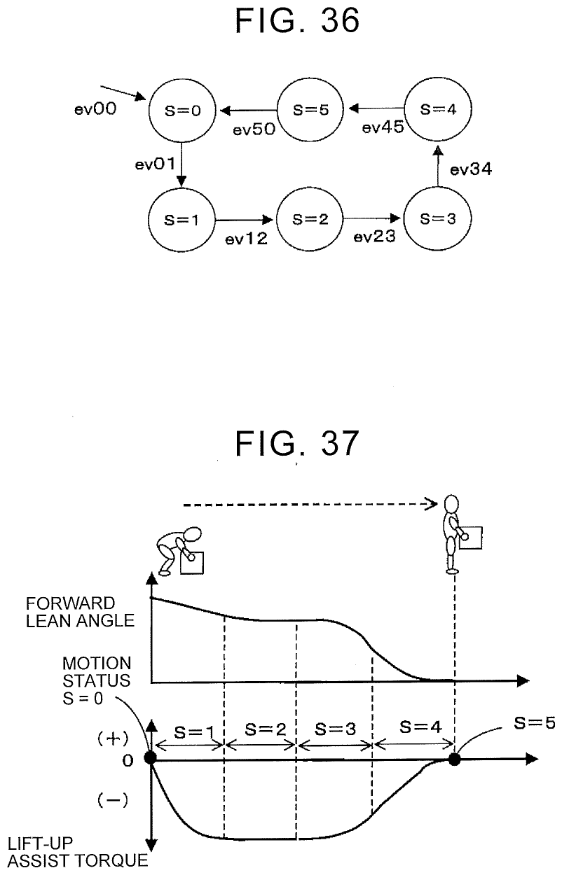

In the process of SU000, the controller 61 advances the process to step SU010. In step SU010, the controller 61 executes the process of SS000 (see FIG. 36), and then advances the process to step SU015. The process of SS000 is a process of, as shown in the state transition diagram of FIG. 36, determining a current motion status S where the entire lift-up motion from the start of lift-up to the end of lift-up is divided into motion statuses S of 0 to 5. The details will be described later.

In step SU015, the controller 61 determines whether it is the timing at which the motion status S shifts from 0 to 1. When it is the timing at which the motion status S shifts from 0 to 1 (Yes), the controller 61 advances the process to step SU020; otherwise (No), the controller 61 advances the process to step SU030.

When the controller 61 has advanced the process to step SU020, the controller 61 substitutes zero for each of assumed elapsed time (right) t_{map,R}(t) and assumed elapsed time (left) t_{map,L}(t), and substitutes zero for each of the lift-up assist torque (right) (assist torque command value (right) τ_{s,cmd,R}(t)) and the lift-up assist torque (left) (assist torque command value (left) τ_{s,cmd,L}(t)). After that, the controller 61 advances the process to step SU030.

When the controller 61 has advanced the process to step SU030, the controller 61 determines whether the motion status S determined in step SU020 is 1. When the motion status S is 1 (Yes), the controller 61 advances the process to step SU031; otherwise (No), the controller 61 advances the process to step SU040.

When the controller 61 has advanced the process to step SU031, the controller 61 adds a task period (for example, when the process shown in FIG. 27 is started at intervals of 2 [ms], 2 [ms]) to the assumed elapsed time (right) t_{map,R}(t), adds the task period to the assumed elapsed time (left) t_{map,L}(t), and then advances the process to step SU032. The assumed elapsed time (right) t_{map,R}(t) and the assumed elapsed time (left) t_{map,L}(t) each indicate (assumed) elapsed time from when the motion status S becomes 1.

In step SU032, the controller 61 determines whether the operation mode is auto-adjust lift-up assist mode. When the operation mode is auto-adjust lift-up assist mode (Yes), the controller 61 advances the process to step SU033R; otherwise (No), the controller 61 advances the process to step SU034.

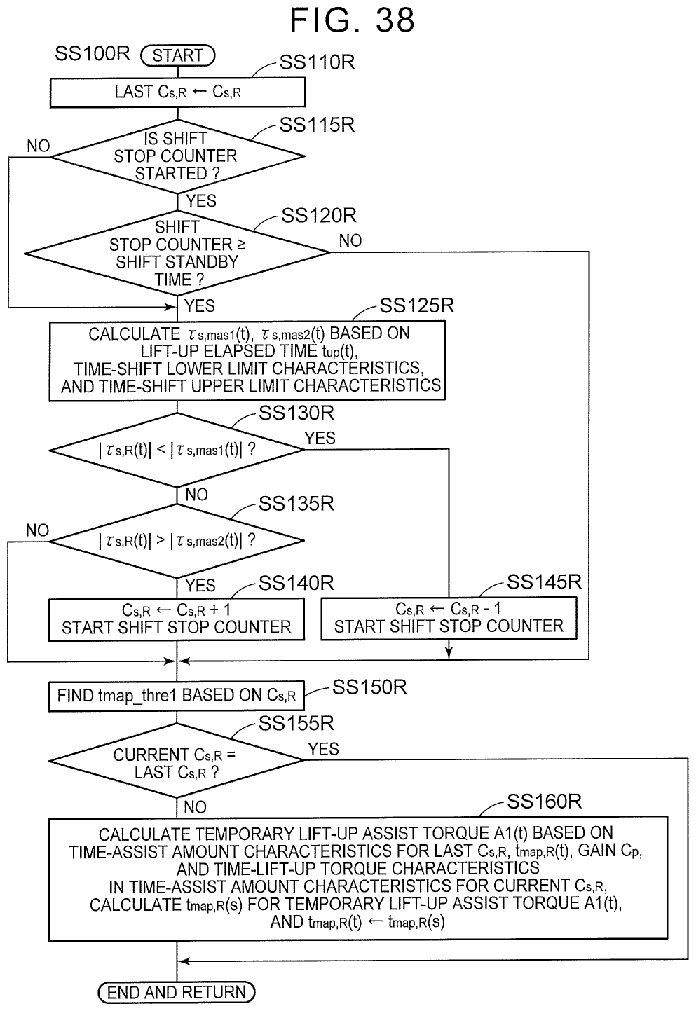

When the controller 61 has advanced the process to step SU033R, the controller 61 executes the process of SS100R (see FIG. 38), and then advances the process to step SU033L. The process of SS100R (see FIG. 38) is a process of changing or keeping the rate of increase (right) C_{s,R} and the assumed elapsed time (right) t_{map,R}(t). The process of SS100L that is a process of changing or keeping the rate of increase (left) C_{s,L} and the assumed elapsed time (left) t_{map,L}(t) is similar to the process of SS100R, so the description thereof is omitted. In step SU033L, the controller 61 executes the process of SS100L, and then advances the process to step SU034. The details of the process of step SS100R will be described later.

In step SU034, the controller 61 determines whether the rate of increase (right) C_{s,R} is equal to the rate of increase (left) C_{s,L}. When the rate of increase (right) C_{s,R} is equal to the rate of increase (left) C_{s,L} (Yes), the controller 61 advances the process to step SU037R; otherwise (No), the controller 61 advances the process to step SU035.

When the controller 61 has advanced the process to step SU035, the controller 61 determines whether the rate of increase (right) C_{s,R} is greater than the rate of increase (left) C_{s,L}. When the rate of increase (right) C_{s,R} is greater than the rate of increase (left) C_{s,L} (Yes), the controller 61 advances the process to step SU036A; otherwise (No), the controller 61 advances the process to step SU036B.

When the controller 61 has advanced the process to step SU036A, the controller 61 substitutes the rate of increase (right) C_{s,R} for the rate of increase (left) C_{s,L}, and then advances the process to step SU037R.

When the controller 61 has advanced the process to step SU036B, the controller 61 substitutes the rate of increase (left) C_{s,L} for the rate of increase (right) C_{s,R}, and then advances the process to step SU037R.

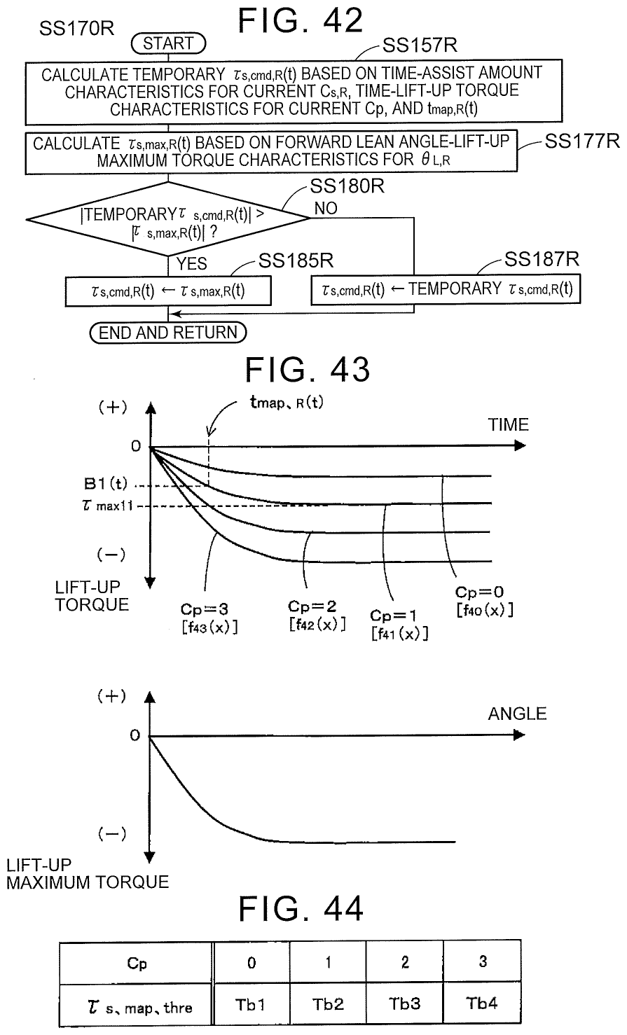

When the controller 61 has advanced the process to step SU037R, the controller 61 executes the process of SS170R (see FIG. 42), and then advances the process to step SU037L. The process of SS170R (see FIG. 42) is a process of finding a lift-up assist torque (right) (assist torque command value (right) τ_{s,cmd,R}(t)) when Motion status S=1. The process of SS170L that is a process of finding a lift-up assist torque (left) (assist torque command value (left) τ_{s,cmd,L}(t)) when Motion status S=1 is similar to the process of SS170R, so the description thereof is omitted. In step SU037L, the controller 61 executes the process of SS170L, ends the process and returns (the controller 61 advances the process to step S060R of FIG. 27). The details of the process of step SS170R will be described later.

When the controller 61 has advanced the process to step SU040, the controller 61 determines whether the motion status S determined in step SU020 is 2. When the motion status S is 2 (Yes), the controller 61 advances the process to step SU041; otherwise (No), the controller advances the process to step SU050.

When the controller 61 has advanced the process to step SU041, the controller 61 determines whether the last motion status S is 1. When the last motion status S is 1 (Yes), the controller 61 advances the process to step SU042; otherwise (No), the controller 61 advances the process to step SU047.

When the controller 61 has advanced the process to step SU042, the controller 61 substitutes zero for each of the assumed elapsed time (right) t_{map,R}(t) and the assumed elapsed time (left) t_{map,L}(t), and then advances the process to step SU047. The process of step SU042 is a process that is executed when the motion status S makes a transition from 1 to 2.

When the controller 61 has advanced the process to step SU047, the controller 61 finds |Maximum value| for the gain Cₚ based on the gain Cₚ and time-lift-up torque characteristics (see FIG. 43). The controller 61 substitutes the found maximum value for each of the lift-up assist torque (right) (assist torque command value (right) τ_{s,cmd,R}(t)) and the lift-up assist torque (left) (assist torque command value (left) τ_{s,cmd,L}(t)), and then ends the process and returns (the controller 61 advances the process to step S060R of FIG. 27). For example, when Gain Cₚ=1, the characteristics of f41(x) for Cₚ=1 in FIG. 43 are used, and τmax11 that is the maximum value of f41(x) is the maximum value to be found. As shown in FIG. 43, the time-lift-up torque characteristics (one of basic lift-up characteristics) are prepared for each gain Cₚ. The controller 61 changes the basic lift-up characteristics for the gain Cₚ.

When the controller 61 has advanced the process to step SU050, the controller 61 determines whether the motion status S determined in step SU020 is 3. When the motion status S is 3 (Yes), the controller 61 advances the process to step SU051; otherwise (No), the controller 61 advances the process to step SU060.

When the controller 61 has advanced the process to step SU051, the controller 61 finds a maximum value for the gain Cₚ based on the gain Cₚ and the time-lift-up torque characteristics (see FIG. 43). The controller 61 substitutes the found maximum value for each of a temporary lift-up assist torque (right) (temporary τ_{s,cmd,R}(t)) and a temporary lift-up assist torque (left) (temporary τ_{s,cmd,L}(t)), and then advances the process to step SU057. For example, when Gain Cₚ=1, the characteristics of f41(x) for Cₚ=1 in FIG. 43 are used, and τmax11 that is the maximum value of |f41(x)| is the maximum value to be found.

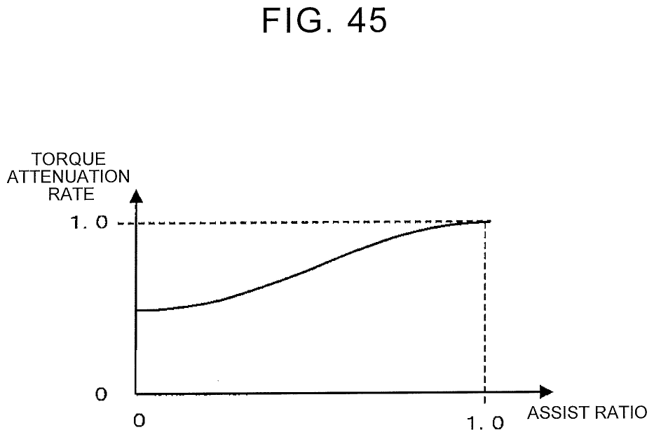

In step S057, the controller 61 finds a torque attenuation rate (right) τ_{d,R} based on the gain Cₚ, the amount of change in user torque (right) τ_{S,R}(t), and assist ratio-torque attenuation rate characteristics (see FIG. 45). Similarly, the controller 61 finds a torque attenuation rate (left) τ_{d,L} based on the gain Cₚ, the amount of change in user torque (left) τ_{S,L}(t), and the assist ratio-torque attenuation rate characteristics (see FIG. 45). Then, the controller 61 finds an assist torque command value (right) τ_{s,cmd,R}(t) by using the following mathematical expression (7) and stores the assist torque command value (right) τ_{s,cmd,R}(t), and finds an assist torque command value (left) τ_{s,cmd,L}(t) by using the following mathematical expression (8) and stores the assist torque command value (left) τ_{s,cmd,L}(t). After that, the controller 61 ends the process and returns (the controller 61 advances the process to step S060R of FIG. 27).

Assist torque command value(right)τ_{s,cmd,R}(t)=Temporary τ_{s,cmd,R}(t)·Torque attenuation rate(right)τ_{d,R} (7)

Assist torque command value(left)τ_{s,cmd,L}(t)=Temporary τ_{s,cmd,L}(t)·Torque attenuation rate(left)τ_{d,L} (8)

For example, when Gain Cₚ=1, the controller 61 finds an attenuation coefficient τ_{s,map,thre}=Tb2 based on gain-attenuation coefficient characteristics shown in FIG. 44. Then, the controller 61 calculates an assist ratio (right) by using the following mathematical expression (9), and calculates an assist ratio (left) by using the following mathematical expression (10).

Assist ratio(right)=[τ_{s,map,thre}−Amount of change in user torque(right)τ_{S,R}(t)]/τ_{s,map,thre} (9)

Assist ratio(left)=[τ_{s,map,thre}−Amount of change in user torque(left)τ_{S,L}(t)]/τ_{s,map,thre} (10)

After that, the controller 61 finds a torque attenuation rate (right) t_{d,R} based on the assist ratio (right) and the assist ratio-torque attenuation rate characteristics (see FIG. 45), and finds a torque attenuation rate (left) τ_{d,L} based on the assist ratio (left) and the assist ratio-torque attenuation rate characteristics (see FIG. 45). Subsequently, the controller 61 stores Temporary τ_{s,cmd,R}(t)·Torque attenuation rate (right) τ_{d,R} in the lift-up assist torque (right) (assist torque command value (right) τ_{s,cmd,R}(t)), and stores Temporary τ_{s,cmd,L}(t)·Torque attenuation rate (left) τ_{d,L} in the lift-up assist torque (left) (assist torque command value (left) τ_{s,cmd,L}(t)).

When the controller 61 has advanced the process to step SU060, the controller 61 determines whether the motion status S determined in step SU020 is 4. When the motion status S is 4 (Yes), the controller 61 advances the process to step SU061; otherwise (No), the controller 61 advances the process to step SU077.

When the controller 61 has advanced the process to step SU061, the controller 61 adds a task period (for example, when the process shown in FIG. 27 is started at intervals of 2 [ms], 2 [ms]) to the assumed elapsed time (right) t_{map,R}(t), adds the task period to the assumed elapsed time (left) t_{map,L}(t), and then advances the process to step SU062. The assumed elapsed time (right) t_{map,R}(t) and the assumed elapsed time (left) t_{map,L}(t) each indicate assumed elapsed time from when the motion status S becomes 4.

In step SU062, the controller 61 substitutes current τ_{s,cmd,R}(t) for last τ_{s,cmd,R}(t−1), and substitutes current τ_{s,cmd,L}(t) for last τ_{s,cmd,L}(t−1), and then advances the process to step SU067.

In step SU067, the controller 61 finds an assist torque command value (right) τ_{s,cmd,R}(t) by using the following mathematical expression (11) and stores the assist torque command value (right) τ_{s,cmd,R}(t), and finds an assist torque command value (left) τ_{s,cmd,L}(t) by using the following mathematical expression (12) and stores the assist torque command value (left) τ_{s,cmd,L}(t). The attenuation coefficient K1 is a preset coefficient and is set to, for example, 0.9. Then, the controller 61 ends the process and returns (the controller 61 advances the process to step S060R of FIG. 27).

Assist torque command value(right)τ_{s,cmd,R}(t)=K1·Last τ_{s,cmd,R}(t−1) (11)

Assist torque command value(left)τ_{s,cmd,L}(t)=K1·Last τ_{s,cmd,L}(t−1) (12)

When the controller 61 has advanced the process to step SU077, the controller 61 finds an assist torque command value (right) τ_{s,cmd,R}(t) by using the following mathematical expression (13) and stores the assist torque command value (right) τ_{s,cmd,R}(t), and finds an assist torque command value (left) τ_{s,cmd,L}(t) by using the following mathematical expression (14) and stores the assist torque command value (left) τ_{s,cmd,L}(t). Then, the controller 61 ends the process and returns (the controller 61 advances the process to step S060R of FIG. 27).

Assist torque command value(right)τ_{s,cmd,R}(t)=0 (13)

Assist torque command value(left)τ_{s,cmd,L}(t)=0 (14)

As described above, during lift-up work, the controller 61 causes the motion status S to make a transition from 0 to 5 sequentially for a lift-up status, and finds a lift-up assist torque (right) (assist torque command value (right) τ_{s,cmd,R}(t)) and a lift-up assist torque (left) (assist torque command value (left) τ_{s,cmd,L}(t)) in accordance with a calculation method set in advance in association with each of the motion statuses S.

Next, the details of the process of SS000 that is step SU020 shown in FIG. 35 will be described with reference to FIG. 36. In SS000, the controller 61 determines any one of the motion statuses S among 0 to 5 for a lift-up status at lift-up work of the user. As shown in FIG. 37, the motion statuses S include Motion status S=0 where the user starts bending forward from an erect state (state where the user finished forward bending of the last work) and starts lift-up motion, Motion state S=1 into which the motion status S makes a transition after the start of lift-up motion, Motion status S=2 where the user is lifting up baggage, Motion status S=3, 4 where the user gradually reduces the forward lean angle, and Motion status S=5 where the user has finished lifting up baggage and in an erect state. The motion status S is set in association with a lift-up status including at least one of the assumed elapsed time (right) t_{map,R}(t), the assumed elapsed time (left) t_{map,L}(t), the link angle (right) (forward lean angle) θ_{L,R}(t), the link angle (left) (forward lean angle) θ_{L,L}(t), the amount of change in user torque (right) τ_{S,R}(t), and the amount of change in user torque (left) τ_{S,L}(t).

Hereinafter, the procedure of determining the motion status S will be described with reference to the state transition diagram shown in FIG. 36. As shown in FIG. 36, in response to event ev00 that lift-up has been started, the controller 61 determines that the motion status S is 0. Determination as to whether lift-up has been started may be made based on the link angle (right) θ_{L,R}(t), the link angle (left) θ_{L,L}(t), the amount of change in link angle (right) Δθ_{L,R}(t), the amount of change in link angle (left) Δθ_{L,L}(t), the amount of change in user torque (right) τ_{S,R}(t), the amount of change in user torque (left) τ_{S,L}(t), or other information. When Motion status S=0, the controller 61 causes the motion status S to make a transition from 0 to 1 upon detection of event ev01. Event ev01 is “normal time”, and, as shown in step SU015 of FIG. 35, after the controller 61 sets the motion status S to 0, the controller 61 causes the motion status S to make a transition into 1 without any condition.

When Motion status S=1, the controller 61 causes the motion status S to make a transition from 1 to 2 upon detection of event ev12. When event ev12 is not detected, the controller 61 keeps the motion status S at 1. Event ev12 occurs, for example, when Assumed elapsed time (right) t_{map,R}(t)≥t_{map,thre1} (right) is satisfied or when Assumed elapsed time (left) t_{map,L}(t)≥t_{map,thre1} (left) is satisfied, or when any one of the link angle (right) (forward lean angle) θ_{L,R}(t) and the link angle (left) (forward lean angle) θ_{L,L}(t) becomes a forward lean angle indicating that the end of lift-up work is close. t_{map,thre1} (right) is determined based on the rate of increase (right) Cs and rate of increase-transition time characteristics (see FIG. 40). t_{map,thre1} (left) is determined based on the rate of increase (left) C_{s,L} and the rate of increase-transition time characteristics (see FIG. 40).

When Motion status S=2, the controller 61 causes the motion status S to make a transition from 2 to 3 upon detection of event ev23. When event ev23 is not detected, the controller 61 keeps the motion status S at 2. Event ev23 occurs, for example, when the amount of change in user torque (right) τ_{S,R}(t) or the amount of change in user torque (left) τ_{S,L}(t) becomes a relatively small amount indicating that the end of lift-up work is close, or when the link angle (right) (forward lean angle) θ_{L,R}(t) or the link angle (left) (forward lean angle) θ_{L,L}(t) becomes a forward lean angle indicating that the end of lift-up work is close.

When Motion status S=3, the controller 61 causes the motion status S to make a transition from 3 to 4 upon detection of event ev34. When event ev34 is not detected, the controller 61 keeps the motion status S at 3. Event ev34 occurs, for example, when Amount of change in user torque (right) τ_{S,R}(t)≥τ_{s,map,thre} or when Amount of change in user torque (left) τ_{S,L}(t)≥τ_{s,map,thre} or when the link angle (right) (forward lean angle) θ_{L,R}(t) or the link angle (left) (forward lean angle) θ_{L,L}(t) becomes a forward lean angle indicating that the end of lift-up work is close. τ_{s,map,thre} is determined based on the gain Cₚ and the gain-attenuation coefficient characteristics (see FIG. 44).

When Motion status S=4, the controller 61 causes the motion status S to make a transition from 4 to 5 upon detection of event ev45. When event ev45 is not detected, the controller 61 keeps the motion status S at 4. Event ev45 occurs, for example, when Assumed elapsed time (right) t_{map,R}(t)≥Status determination time t41 (for example, about 0.15 [sec]) or when Assumed elapsed time (left) t_{map,L}(t)≥Status determination time t41 (for example, about 0.15 [sec]).

When Motion status S=5, the controller 61 causes the motion status S to make a transition from 5 to 0 upon detection of event ev50. When event ev50 is not detected, the controller 61 keeps the motion status S at 5. Event ev50 is a start of lift-up work. After lift-up work is finished, the motion status S returns to 0.

Next, the details of the process of SS100R that is step SU033R shown in FIG. 35 will be described with reference to FIG. 38. In SS100R, the controller 61 automatically shifts the rate of increase (right) C_{s,R} into an appropriate value among −1 to 4 for lift-up motion of the user. The process of SS100R shows a procedure to automatically shift the rate of increase (right) C_{s,R}. A procedure of SS100L (see FIG. 35) to automatically shift the rate of increase (left) C_{s,L} is similar to the that of SS100R, so the description thereof is omitted.

In the process of SS100R, the controller 61 advances the process to step SS11R. In step SS10R, the controller 61 stores a current rate of increase (right) C_{s,R} in last C_{s,R}, and then advances the process to step SS115R.

In step SS115R, the controller 61 determines whether a shift stop counter is active. When the shift stop counter is active (Yes), the controller 61 advances the process to step SS120R; otherwise (No), the controller 61 advances the process to step SS125R. The shift stop counter is a counter that is activated at the time when the rate of increase (right) C_{s,R} has been shifted (changed) in step SS140R or step SS145R.

When the controller 61 has advanced the process to step SS120R, the controller 61 determines whether the shift stop counter is longer than or equal to shift standby time. When the shift stop counter is longer than or equal to the shift standby time (Yes), the controller 61 advances the process to step SS125R; otherwise (No), the controller 61 advances the process to step SS150R.

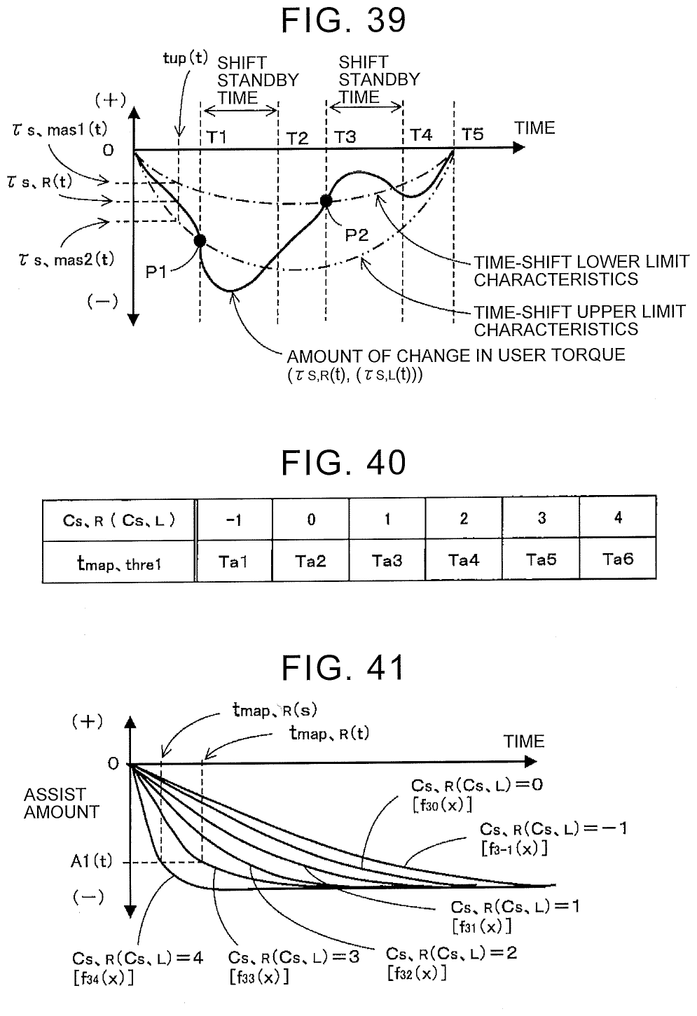

When the controller 61 has advanced the process to step SS125R, the controller 61 finds a shift lower limit τ_{s,mas1}(t) for current lift-up elapsed time tᵤₚ(t) based on the lift-up elapsed time tᵤₚ(t) and time-shift lower limit characteristics (see FIG. 39). The controller 61 also finds a shift upper limit τ_{s,mas2}(t) for current lift-up elapsed time tᵤₚ(t) based on the current rate of increase (right) C_{s,R}, the lift-up elapsed time tᵤₚ(t), and time-shift upper limit characteristics (see FIG. 39). The lift-up elapsed time tᵤₚ(t) is elapsed time from timing at which lift-up is started (the motion status S makes a transition from 0 to 1). Then, the controller 61 advances the process to step SS130R. The example shown in FIG. 39 shows an example of a state where |Amount of change in user torque (right) τ_{S,R}(t)|>|Shift upper limit τ_{s,mas2}(t)| is satisfied at time T1 (at the position of P1) and an example of a state where |Amount of change in user torque (right) τ_{S,R}(t)|<|Shift upper limit τ_{s,mas1}(t)| is satisfied at time T3 (at the position of P2).

In step SS130R, the controller 61 determines whether |Amount of change in user torque (right) τ_{S,R}(t)| is less than |Shift lower limit τ_{s,mas1}(t)|. When |Amount of change in user torque (right) τ_{S,R}(t)| is less than |Shift lower limit τ_{s,mas1}(t)| (Yes), the controller 61 advances the process to step SS145R; otherwise (No), the controller 61 advances the process to step SS135R.

When the controller 61 has advanced the process to step SS135R, the controller 61 determines whether |Amount of change in user torque (right) τ_{S,R}(t)| is greater than |Shift upper limit τ_{s,mas2}(t)|. When |Amount of change in user torque (right) τ_{S,R}(t)| is greater than |Shift upper limit τ_{s,mas2}(t)| (Yes), the controller 61 advances the process to step SS140R; otherwise (No), the controller 61 advances the process to step SS150R.

When the controller 61 has advanced the process to step SS140R, the controller 61 increments the rate of increase (right) C_{s,R} by one (however, the maximum value is set to 4), activates the shift stop counter, and then advances the process to step SS150R.

When the controller 61 has advanced the process to step SS145R, the controller 61 decrements the rate of increase (right) C_{s,R} by one (however, the minimum value is set to −1), activates the shift stop counter, and then advances the process to step SS150R.

When the controller 61 has advanced the process to step SS150R, the controller 61 finds t_{map,thre1} (right) based on the rate of increase (right) C_{s,R} and the rate of increase-transition time characteristics (see FIG. 40), and then advances the process to step S155R. t_{map,thre1} (right) is used in determining the motion status (determining whether the motion status has made a transition from 1 to 2), or the like.

In step SS155R, the controller 61 determines whether the current rate of increase (right) C_{s,R} is equal to last C_{s,R} (see step SS110R). When the current rate of increase (right) C_{s,R} is equal to last C_{s,R} (Yes), the controller 61 ends the process and returns (the controller 61 returns to step SU033L of FIG. 35); otherwise (No), the controller 61 advances the process to step SS160R.

When the controller 61 has advanced the process to step SS160R, the controller 61 calculates a temporary lift-up assist torque A1(t) based on last C_{s,R}, the assumed elapsed time (right) t_{map,R}(t), time-assist amount characteristics (see FIG. 41), the gain Cₚ, and the time-lift-up torque characteristics (see FIG. 43). For example, when Last C_{s,R}=3, the controller 61 calculates a temporary lift-up assist torque A1(t) from f33(x) for C_{s,R}=3 and the assumed elapsed time (right) t_{map,R}(t), as shown in FIG. 41. As shown in FIG. 41, the time-assist amount characteristics (one of the basic lift-up characteristics) are prepared for each of the rate of increase (right) C_{s,R} and the rate of increase (left) C_{s,L} and the controller 61 changes the basic lift-up characteristics for the rate of increase (right) C_{s,R} or the rate of increase (left) C_{s,L}.

Then, the controller 61 calculates a torque deviation reduction assumed elapsed time t_{map,R}(S) for the temporary lift-up assist torque A1(t) based on the current rate of increase (right) C_{s,R}, the time-assist amount characteristics (see FIG. 41), the gain Cₚ, and the time-lift-up torque characteristics (see FIG. 43), and substitutes (rewrites) the calculated torque deviation reduction assumed elapsed time t_{map,R}(S) for the assumed elapsed time (right) t_{map,R}(t). For example, when the current rate of increase (right) C_{s,R}=4, the controller 61 calculates a torque deviation reduction assumed elapsed time t_{map,R}(S) from f34(x) for C_{s,R}=4 and the temporary lift-up assist torque A1(t) as shown in FIG. 41, and substitutes the torque deviation reduction assumed elapsed time t_{map,R}(s) for the assumed elapsed time (right) t_{map,R}(t). Then, the controller 61 ends the process and returns (the controller 61 returns to step SU033L of FIG. 35). Rewriting of the assumed elapsed time (right) t_{map,R}(t) may be regarded as, when the motion status S has made a transition into a predetermined motion status S (in this case, Motion status S=1), shift-time torque deviation reduction correction of reducing the deviation between the lift-up assist torque (temporary lift-up assist torque A1(t)) found based on the selected basic lift-up characteristics (the time-assist amount characteristics (see FIG. 41) for the last rate of increase (right) C_{s,R}) and the currently selected basic lift-up characteristics (the time-assist amount characteristics (see FIG. 41) for the current rate of increase (right) C_{s,R}).

In the above description, the time-assist amount characteristics (see FIG. 41), the time-lift-up torque characteristics, and the forward lean angle-lift-up maximum torque characteristics (see FIG. 43) may be regarded as a plurality of basic lift-up characteristics in which a lift-up assist torque that is a torque in the lift-up direction is set. Then, the controller 61 selects appropriate basic lift-up characteristics, finds a lift-up assist torque based on the selected basic lift-up characteristics, and drives the actuator unit based on an assist torque by using the found lift-up assist torque as the assist torque.

Next, the details of the process of SS170R that is step SU037R shown in FIG. 35 will be described with reference to FIG. 42. In SS170R, the controller 61 finds a lift-up assist torque (right) (assist torque command value (right)) τ_{s,cmd,R}(t). The process of SS170R shows a procedure to find a lift-up assist torque (right) (assist torque command value (right)) τ_{s,cmd,R}(t). A procedure of SS170L (see FIG. 35) to find a lift-up assist torque (left) (assist torque command value (left)) τ_{s,cmd,L}(t) is similar to that of SS170R, so the description thereof is omitted.

In the process of SS170R, the controller 61 advances the process to step SS175R. In step SS175R, the controller 61 calculates temporary τ_{s,cmd,R}(t) based on the current rate of increase (right) C_{s,R}, the assumed elapsed time (right) t_{map,R}(t), the gain Cₚ, the time-assist amount characteristics (see FIG. 41), and the time-lift-up torque characteristics (see FIG. 43), and then advances the process to step SS177R. For example, when the current rate of increase (right) C_{s,R}=3, the controller 61 stores an assist torque A1(t) found from f33(x) for C_{s,R}=3 and the assumed elapsed time (right) t_{map,R}(t) as shown in FIG. 41 in temporary τ_{s,cmd,R}(t).

In step SS177R, the controller 61 calculates a torque upper limit (right) τ_{s,max,R}(t) based on the forward lean angle and the forward lean angle-lift-up maximum torque characteristics (see FIG. 43), and then advances the process to step SS180R. For example, the controller 61 stores a lift-up maximum torque B1(t) found from the forward lean angle-lift-up maximum torque characteristics shown in FIG. 43 and the link angle (right) (forward lean angle) θ_{L,R}(t) in the torque upper limit (right) τ_{s,max,R}(t). Lift-up torque is limited by using the forward lean angle-lift-up maximum torque characteristics so that the lift-up torque does not excessively increases when the forward lean angle is small.

In step SS180R, the controller 61 determines whether |Temporary τ_{s,cmd,R}(t)| is greater than |Torque upper limit (right) τ_{s,max,R}(t)|. When |Temporary τ_{s,cmd,R}(t)| is greater than |Torque upper limit (right) τ_{s,max,R}(t)| (Yes), the controller 61 advances the process to step SS185R; otherwise (No), the controller 61 advances the process to step SS187R.

When the controller 61 has advanced the process to step SS185R, the controller 61 stores the torque upper limit (right) τ_{s,max,R}(t) in the lift-up assist torque (right) (assist torque command value (right) τ_{s,cmd,R}(t)), and then ends the process and returns (the controller 61 returns to step SU037L of FIG. 35).

When the controller 61 has advanced the process to step SS187R, the controller 61 stores temporary τ_{s,cmd,R}(t) in the lift-up assist torque (right) (assist torque command value (right) τ_{s,cmd,R}(t)), and then ends the process and returns (the controller 61 returns to step SU037L of FIG. 35).

As described above, the assist device 1 described in the present embodiment has a simple configuration and makes it easy for the user to wear. In addition, assist control for bring-down work and assist control for lift-up work both are simple control, and the assist device 1 is able to appropriately assist at work for lifting up baggage and work for bringing down baggage.

The assist device 1B (see FIG. 46) of the second embodiment that will be described below differs from the assist device 1 (see FIG. 1) of the first embodiment in that the torque generation units, such as the electric motors, are accommodated in a backpack part 37B (see FIG. 46, which may be regarded as a control box). In the assist device 1B of the second embodiment, the torque generation units, the controller 61, and the power supply unit 63 (which may be regarded as a battery) are accommodated in the backpack part 37B. That is, the torque generation units respectively mounted at the right hip region and left hip region of the user in the first embodiment are moved into the backpack part 37B in the second embodiment. Therefore, the number of members that are mounted at the right hip region and left hip region of the user reduces, with the result that no such members interfere with movements of the arms during lift-up motion or bring-down motion of the user. In addition, in the first embodiment, a relatively large cover and other parts that accommodate the torque generation unit are required at each of the right hip region and the left hip region; whereas, in the second embodiment, the torque generation units are accommodated in the backpack part 37B. Therefore, the cover and other parts can be shared, so the lightweight feature is improved.

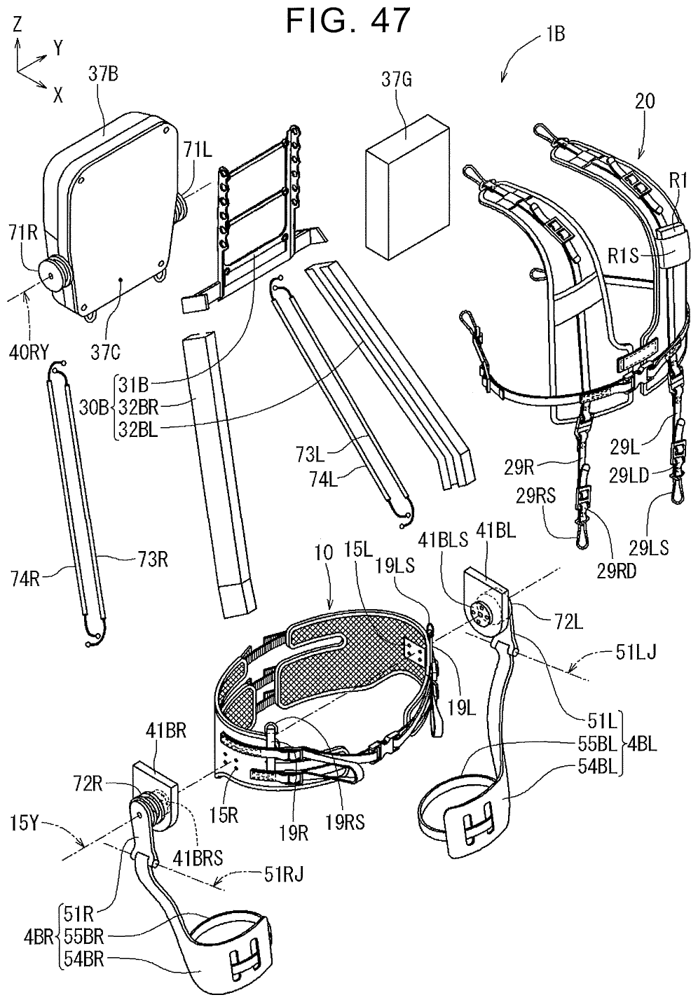

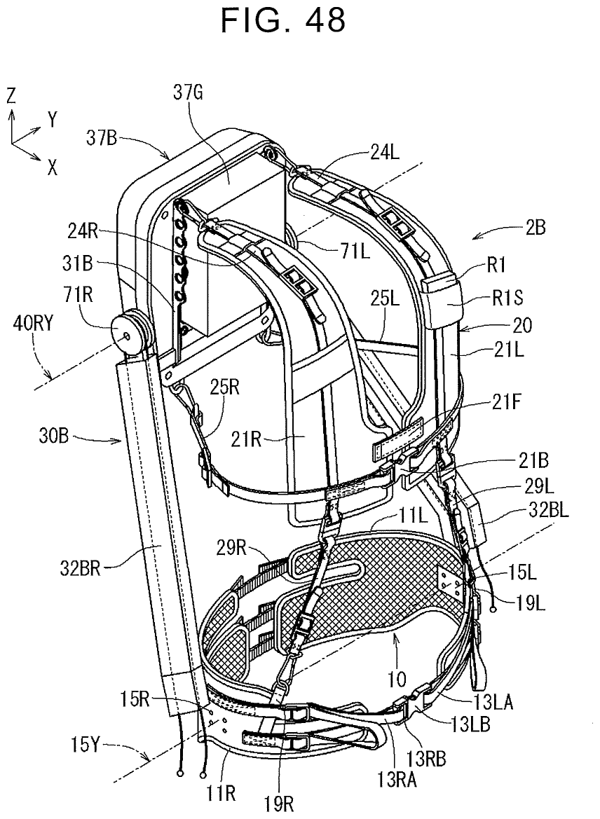

Hereinafter, the overall structure of the assist device 1B of the second embodiment will be described with reference to FIG. 46 and FIG. 47. A procedure of the controller 61 is similar to that of the first embodiment, so the description thereof is omitted. FIG. 46 shows the overall appearance of the assist device 1B of the second embodiment. FIG. 47 shows an exploded perspective view of the assist device 1B shown in FIG. 46. The right armpit belt 25R and the left armpit belt 25L in FIG. 46 may be replaced with the close contact belt 25RL as shown in FIG. 2.

As shown in the overall perspective view of FIG. 46 and the exploded perspective view of FIG. 47, the assist device 1B of the second embodiment is made up of the lower back support part 10, the jacket part 20, a frame part 30B, the backpack part 37B, the cushion 37G, a right thigh unit 4BR, a left thigh unit 4BL, and other parts. A body-worn component 2B (see FIG. 48) is worn at least around the hip of the user. Each of the right thigh unit 4BR and the left thigh unit 4BL (see FIG. 47) is mounted on the body-worn component 2B (see FIG. 48) and the thigh of the user, and assists the user with the movement of the thigh relative to the lower back or the movement of the lower back to the thigh. The lower back support part 10, the jacket part 20, and the cushion 37G are the same as those of the assist device 1 (see FIG. 1) of the first embodiment, so the description thereof is omitted. The frame part 30B, the backpack part 37B, the right thigh unit 4BR, and the left thigh unit 4BL that are different from those of the first embodiment will be mainly described.

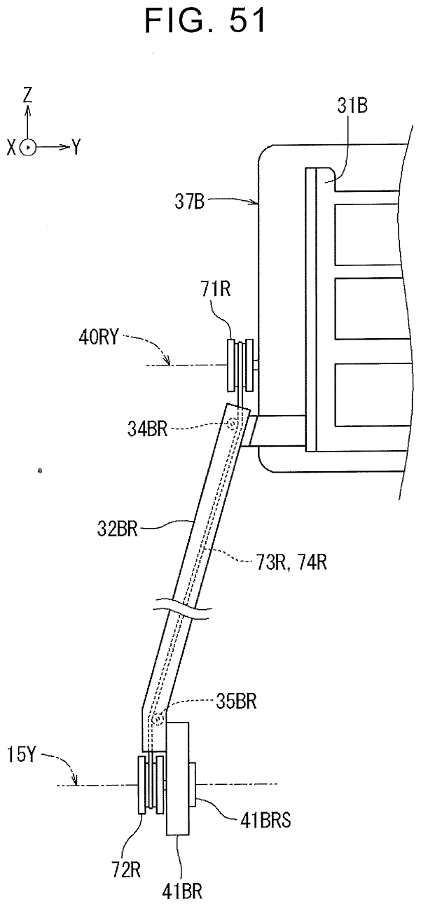

As shown in FIG. 46 and FIG. 47, the frame part 30B is made up of a main frame 31B, a right frame 32BR, a left frame 32BL, and the like. As shown in FIG. 51, a portion of the right frame 32BR near the upper end is fixed to the right lower end of the main frame 31B, and a portion of the right frame 32BR near the lower end is fixed to a right hip base 41BR. Similarly, a portion of the left frame 32BL near the upper end is fixed to the left lower end of the main frame 31B, and a portion of the left frame 32BL near the lower end is fixed to a left hip base 41BL. As shown in FIG. 46, the right thigh unit 4BR (left thigh unit 4BL) is mounted on the lower back support part 10 via the right hip base 41BR (left hip base 41BL) mounted on the right hip portion (left hip portion) of the lower back support part 10. As shown in FIG. 46, the right hip base 41BR (left hip base 41BL) and the backpack part 37B are connected via the right frame 32BR (left frame 32BL).

The right frame 32BR (left frame 32BL) is made from a tubular metal, resin, or other materials. As shown in the cross section of FIG. 50, for example, part of the side facing the user is removed for weight reduction. Each of the right frame 32BR and the left frame 32BL has a structure such that the stiffness in a right and left direction (Y-axis direction in FIG. 50) with respect to the user wearing the assist device 1B is lower than the stiffness in a front and rear direction (X-axis direction in FIG. 50) with respect to the user. Thus, each of the right frame 32BR and the left frame 32BL elastically deforms to curve in the right and left direction for the hip width of the wearing user.

A coupling portion 41BRS and a right driven pulley 72R are mounted on the right hip base 41BR. The coupling portion 41BRS pivots about the assumed pivot axis 15Y. The right driven pulley 72R pivots about the assumed pivot axis 15Y. The coupling portion 41BRS is fixed via the mounting holes 15R of the lower back support part 10, so the coupling portion 41BRS is connected to the right hip portion of the lower back support part 10. Thus, the right hip base 41BR pivots about the assumed pivot axis 15Y relative to the lower back support part 10. The left hip base 41BL also has a similar structure and connection. The left hip base 41BL connected to the left hip portion of the lower back support part 10 pivots about the assumed pivot axis 15Y relative to the lower back support part 10.

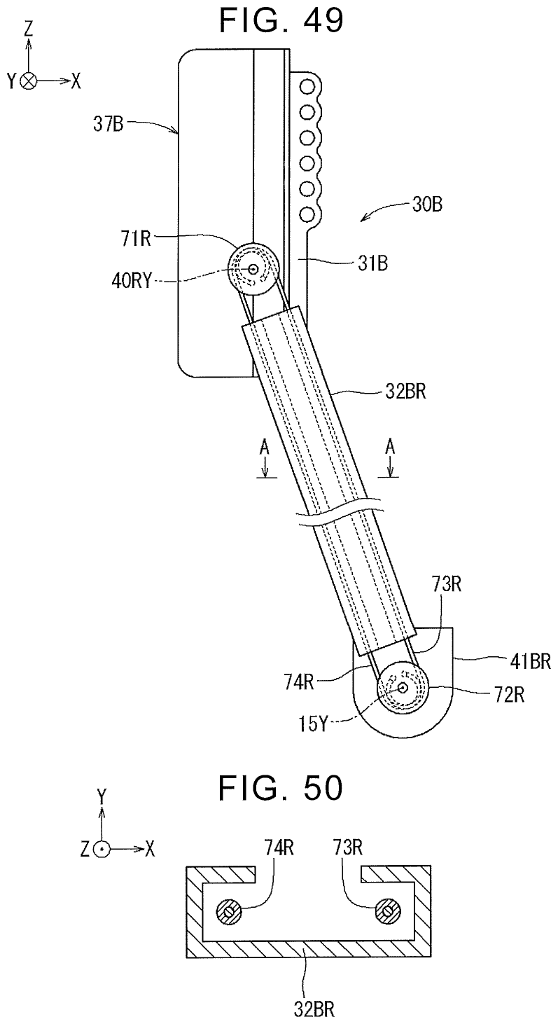

As shown in FIG. 49 and FIG. 51, a right drive pulley 71R (left drive pulley 71L) that pivots about a pivot axis 40RY is disposed at a portion extended from the upper end of the tubular right frame 32BR (left frame 32BL). The right driven pulley 72R (left driven pulley 72L) that pivots about the assumed pivot axis 15Y is disposed at a portion extended from the lower end of the tubular right frame 32BR (left frame 32BL).

As shown in FIG. 49 and FIG. 51, cables 73R, 74R are inserted in the right frame 32BR. One end of the cables 73R, 74R are connected to the right drive pulley 71R, and the other ends of the cables 73R, 74R are connected to the right driven pulley 72R. Tension is applied to the cables 73R, 74R. In FIG. 49, the cable 73R transmits torque in the counter-clockwise direction of the right drive pulley 71R to the right driven pulley 72R. The cable 74R transmits torque in the clockwise direction of the right drive pulley 71R to the right driven pulley 72R. As shown in FIG. 51, a guide roller 34BR is provided in the right frame 32BR. The guide roller 34BR guides the cables 73R, 74R drawn out from the right drive pulley 71R such that the drawn-out direction is perpendicular to the pivot axis 40RY of the right drive pulley 71R. Similarly, a guide roller 35BR is provided in the right frame 32BR. The guide roller 35BR guides the cables 73R, 74R drawn out from the right driven pulley 72R such that the drawn-out direction is perpendicular to the assumed pivot axis 15Y of the right driven pulley 72R. Guide rollers are also provided in the left frame 32BL and are similar to the above-described guide rollers 34BR, 35BR, so the description thereof is omitted. Cables in which wires are inserted through sleeves (cylindrical sheaths) may be used as the cables 73R, 74R or cables having only wires without sleeves may be used as the cables 73R, 74R. The material of the cables is, for example, a metal or a resin. Cables made of various materials may be used.

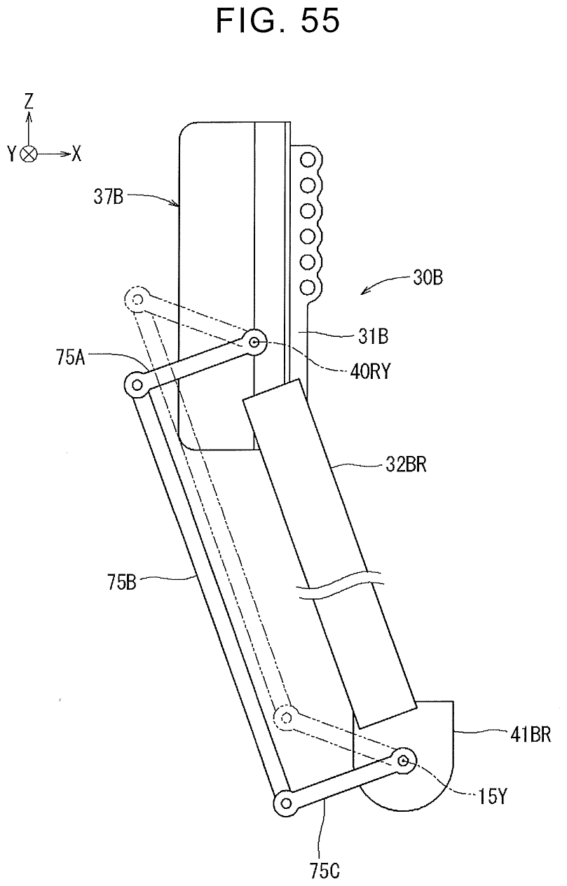

The right drive pulley 71R, the right driven pulley 72R, the right frame 32BR, and the cables 73R, 74R make up a power transmission unit that transmits assist torque generated inside the backpack part 37B to the right thigh unit 4BR. The right drive pulley 71R and the right driven pulley 72R may be regarded as a tension adjustment part that adjusts the tension of each of the cables 73R, 74R. Similarly, the left drive pulley 71L, the left driven pulley 72L, the left frame 32BL, and the cables 73L, 74L make up a power transmission unit that transmits assist torque generated inside the backpack part 37B to the left thigh unit 4BL. The left drive pulley 71L and the left driven pulley 72L may be regarded as a tension adjustment part that adjusts the tension of each of the cables 73L, 74L. As shown in FIG. 55, the right drive pulley 71R, the right driven pulley 72R, the cable 73R, and the cable 74R may be replaced with link members 75A, 75B, 75C that make up a parallel linkage.

As shown in FIG. 46, the right drive pulley 71R is provided to the lower right side of the backpack part 37B. The right drive pulley 71R pivots about the pivot axis 40RY to cause the right thigh unit 4BR to pivot. The left drive pulley 71L is provided to the lower left side of the backpack part 37B. The left drive pulley 71L pivots about the pivot axis 40RY to cause the left thigh unit 4BL to pivot. As shown in FIG. 52, the right torque generation unit 40BR and the left torque generation unit 40BL are accommodated in the backpack part 37B. The right torque generation unit 40BR drives the right drive pulley 71R. The left torque generation unit 40BL drives the left drive pulley 71L. The backpack part 37B in the second embodiment differs from the backpack part 37 of the first embodiment shown in FIG. 24 in that the right torque generation unit 40BR and the left torque generation unit 40BL are added. That is, the controller 61, the motor driver 62, the power supply unit 63 (which may be regarded as the battery), the right torque generation unit 40BR, the left torque generation unit 40BL, and other components are accommodated in the backpack part 37B.

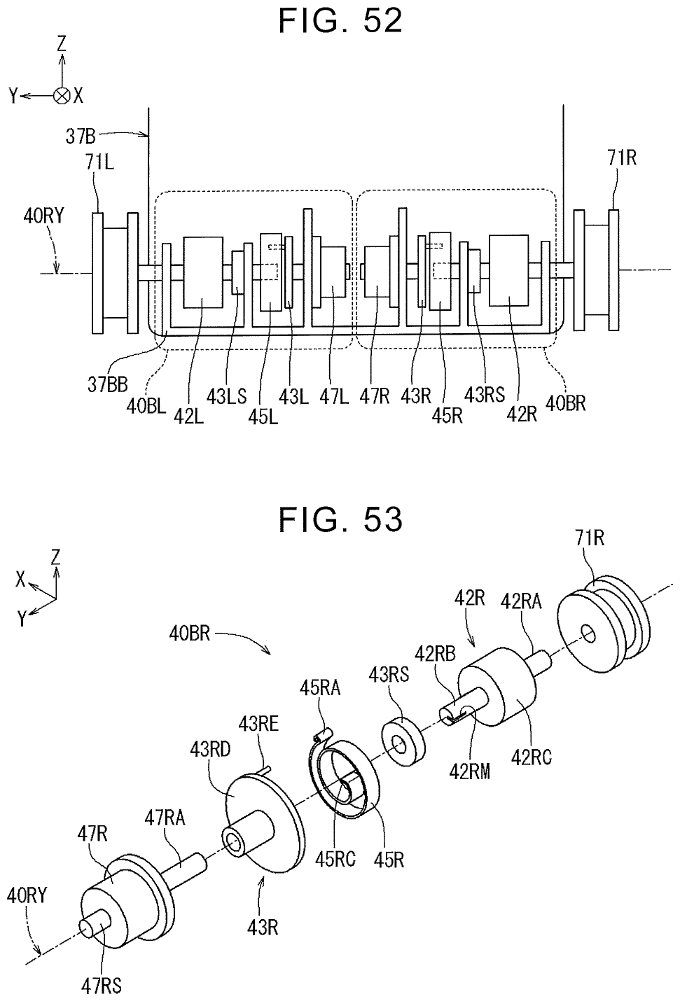

Since the right torque generation unit 40BR and the left torque generation unit 40BL have similar structures and dispositions, the structure and disposition of the right torque generation unit 40BR will be described below. FIG. 52 shows the appearance of the right torque generation unit 40BR and left torque generation unit 40BL accommodated in the backpack part 37B. FIG. 53 shows an exploded perspective view of the right torque generation unit 40BR including the right drive pulley 71R. As shown in FIG. 52, the right torque generation unit 40BR and the left torque generation unit 40BL are supported on a support body 37BB fixed inside the backpack part 37B.

As shown in FIG. 52 and FIG. 53, the right torque generation unit 40BR is made up of the electric motor 47R, a spring support body 43R, a spiral spring 45R, the output link pivot angle detection unit 43RS, the speed reduction gear 42R, and other parts. The configuration of the right torque generation unit 40BR is similar to that of the torque generation unit 40R shown in FIG. 19 and FIG. 20. When the right torque generation unit 40BR shown in FIG. 52 and FIG. 53 are compared with the torque generation unit 40R in which the parts are disposed in parallel as shown in FIG. 19 and FIG. 20, the parts are disposed in series with one another. In addition, the right torque generation unit 40BR shown in FIG. 52 and FIG. 53 is accommodated in the backpack part 37B, so the sub-frame 48R, the cover 41RB, and other parts shown in FIG. 19 and FIG. 20 can be omitted. Therefore, the right torque generation unit 40BR is reduced in weight.

As described above, the electric motor 47R includes the motor rotation angle detection unit 47RS and the output shaft 47RA, and is fixed to the support body 37BB. The output shaft 47RA is fitted into a shaft hole of the spring support body 43R, and pivots together with the spring support body 43R. The spring support body 43R has the flange 43RD. The transmission shaft 43RE is provided near the outer periphery of the flange 43RD. The transmission shaft 43RE is inserted into the cylindrical portion of the outer end portion 45RA of the spiral spring 45R, and supports the outer end portion 45RA. The inner end portion 45RC of the spiral spring 45R is inserted into a groove 42RM of the speed increasing shaft 42RB inserted through the output link pivot angle detection unit 43RS. The output link pivot angle detection unit 43RS is fixed to the support body 37BB. The output link pivot angle detection unit 43RS outputs a detected signal commensurate with a pivot angle of the speed increasing shaft 42RB to the controller 61.

As described above, the speed reduction gear 42R includes the speed reduction gear housing 42RC, the speed reduction shaft 42RA and the speed increasing shaft 42RB. The speed reduction shaft 42RA and the speed increasing shaft 42RB are supported so as to be pivotable relative to the support body 37BB. The right drive pulley 71R is fixed to the speed reduction shaft 42RA. In this way, the electric motor 47R, the spring support body 43R, the spiral spring 45R, the output link pivot angle detection unit 43RS, the speed reduction gear 42R, and the right drive pulley 71R are disposed in series with one another along the pivot axis 40RY.

As shown in FIG. 46, the thigh units include the right thigh unit 4BR and the left thigh unit 4BL. The right thigh unit 4BR is worn on the right thigh of the user. The left thigh unit 4BL is worn on the left thigh of the user.

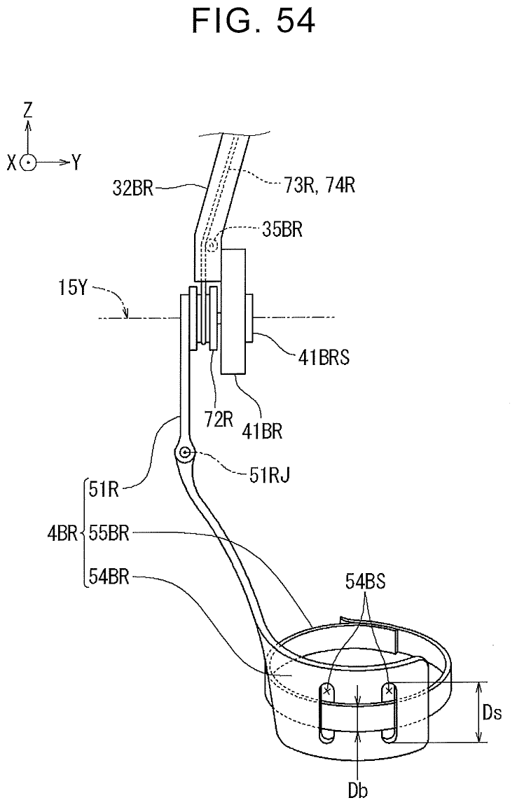

As shown in FIG. 46, FIG. 47, and FIG. 54, the right thigh unit 4BR includes the assist arm 51R, a thigh worn portion 54BR, and a thigh belt 55BR. The thigh worn portion 54BR is pivotable about the pivot axis 51RJ relative to the assist arm 51R. As shown in FIG. 46, the assist arm 51R of the right thigh unit 4BR is fixed to the right driven pulley 72R of the right hip base 41BR connected to the right hip portion of the lower back support part 10, with the result that the assist arm 51R is mounted on the lower back support part 10 via the right hip base 41BR.

As shown in FIG. 46 and FIG. 47, the left thigh unit 4BL includes the assist arm 51L, a thigh worn portion 54BL, and a thigh belt 55BL. The thigh worn portion 54BL is pivotable about a pivot axis 51LJ relative to the assist arm 51L. As shown in FIG. 46, the assist arm 51L of the left thigh unit 4BL is fixed to the left driven pulley 72L of the left hip base 41BL connected to the left hip portion of the lower back support part 10, with the result that the assist arm 51L is mounted on the lower back support part 10 via the left hip base 41BL. The left thigh unit 4BL has a similar structure to that of the right thigh unit 4BR, so the right thigh unit 4BR will be described below, and the description of the left thigh unit 4BL is omitted.

As shown in FIG. 54, the right thigh unit 4BR includes the thigh worn portion 54BR having a surface that contacts with (surface worn on) the right thigh of the user. The thigh worn portion 54BR has a plurality of slits 54BS along a thigh extending direction that is a direction in which the thigh of the user extends. The thigh belt 55BR to be fixedly wound around the right thigh of the user is inserted through the slits 54BS. The thigh belt 55BR has, for example, a hook-and-loop fastener, and can be appropriately and easily wound and fixed to thighs having various diameters.

As shown in FIG. 54, the length Ds of the slits 54BS in the thigh extending direction is greater than the length Db of the thigh belt 55BR in the thigh extending direction. Therefore, at the time when the right thigh unit 4BR worn on the right thigh of the user pivots about the assumed pivot axis 15Y, even when the thigh belt 55BR slides in the thigh extending direction relative to the thigh worn portion 54BR, the thigh belt 55BR is allowed to slide in the slits 54BS along the thigh extending direction. Therefore, no special slide mechanism for sliding the thigh belt 55BR needs to be provided for the thigh worn portion 54BR, so size reduction and weight reduction are further improved.

With the above-described assist device 1B of the second embodiment, in comparison with the assist device 1 of the first embodiment, the torque generation units are accommodated in the backpack part 37B (control box). Therefore, rightward and leftward projections from the hip of the user are further reduced, so the assist device 1B does not interfere with movements of the arms during lift-up motion or bring-down motion of the user. In addition, in comparison with the assist device 1 of the first embodiment, no electric power supply lines are required to be routed from the backpack part 37B to the hip, so assembling during manufacturing is relatively easy. in comparison with the assist device 1 of the first embodiment, the torque generation units respectively disposed at the right and left hip regions are accommodated in the backpack part 37B. Therefore, the number of various parts is reduced and various parts are shared, so size reduction and weight reduction are further improved. In the second embodiment, for the sake of easy illustration, the right drive pulley 71R (left drive pulley 71L) and the right driven pulley 72R (left driven pulley 72L) are exposed. Instead, the right drive pulley 71R (left drive pulley 71L) and the right driven pulley 72R (left driven pulley 72L) each may be covered with a cover or other parts.

The structure, configuration, shape, appearance, procedure, and the like, of each of the assist devices 1, 1A, 1B of the embodiments of the disclosure may be modified, added, or omitted in various forms without departing from the scope of the disclosure. For example, the procedure of the controller is not limited to the flowcharts, and the like, described in the embodiments. In the example of the description of the embodiments, the spiral spring 45R (see FIG. 19) is used. Instead of a spiral spring, a torsion bar or a torsion bar spring may be used.

In the examples of the assist devices 1, 1A, 1B described in the embodiments, the adjusters or buckles are used as belt holding members that hold the belts in a fastened state. Then, connection and release of the belts, and the like, are performed by using the buckles. Instead, connection and release of the belts, and the like, may be performed by using belt holding members different from the buckles. A slack of the belt under tension is prevented by inserting the belt through the adjuster. Instead, a belt holding member other than the adjuster may be used. Alternatively, a belt holding member having the function of both adjuster and buckle may be used.

In the description of the examples of the embodiments, the operating unit R1 has both the set of gain UP control button R1BU and gain DOWN control button R1BD and the set of rate-of-increase UP control button R1CU and rate-of-increase DOWN control button R1CD. Instead, the operating unit R1 may be configured to have at least one of the set of gain UP control button R1BU and gain DOWN control button R1BD and the set of rate-of-increase UP control button R1CU and rate-of-increase DOWN control button R1CD.

In the description of the examples of the embodiments, the backpack parts 37, 37B are disposed on the back of the user; however, the location of the backpack part is not limited to the region of the back. The backpack part just needs to be disposed above the hip of the user. With the assist device 1B of the second embodiment, when the lower back support part 10 is worn around the hip of the user, the lower back support part 10 does not slip and closely contacts with the hip of the user, so a deviation of the assumed pivot axis 15Y of the right (left) hip base 41BR (41BL) reduces. Therefore, the output of the right (left) torque generation unit 40BR (40BL) disposed above the hip of the user is efficiently transmitted to the right (left) thigh unit 4BR (4BL) that pivots about the stably supported assumed pivot axis 15Y. For this reason, a loss of the output of the right (left) torque generation unit 40BR (40BL) is further reduced, and the output is efficiently transmitted to the right (left) thigh unit 4BR (4BL). Hence, the upper body of the user is efficiently raised (the forward lean angle of the upper body relative to the thigh is reduced). Furthermore, when the jacket part 20 is worn on the user, the jacket part 20 does not slip and closely contacts with the chest of the user, so a misalignment of the right (left) torque generation unit 40BR (40BL) disposed above the hip of the user reduces. For this reason, a loss of the output of the right (left) torque generation unit 40BR (40BL) is further more reduced, and the output is efficiently transmitted to the right (left) thigh unit 4BR (4BL). Hence, the upper body of the user is more efficiently raised (the forward lean angle of the upper body relative to the thigh is reduced).

In the assist devices 1, 1A, 1B described in the examples of the embodiments, the operation mode, the gain, the rate of increase, and the like, can be changed with the operating unit R1. Instead, the controller 61 may include the communication unit 64 (see FIG. 24) (that carries out communication by wireless connection or wired connection) and the operation mode, the gain, the rate of increase, and the like, may be allowed to be changed through communication from a smartphone, or other devices. Alternatively, the controller 61 may include the communication unit 64 (see FIG. 24) (that carries out communication by wireless connection or wired connection), the controller 61 may collect various data, and may transmit the collected data to an analysis system at predetermined timing (constantly, at set time intervals, after the end of assist operation, or at other timing). For example, the collected data contains user information and assist information. Examples of the user information include user torque and the posture of the user. The user information is information related to the user. Examples of the assist information include assist torque, the rotation angle (actual motor shaft angle θ_{rM} in FIG. 24) of the electric motor (actuator), output link pivot angle (actual link angle θ_{L} in FIG. 24), operation mode, gain, and rate of increase. The assist information is information related to inputs and outputs of the right and left actuator units. The analysis system may be a system prepared separately from the assist device, and is, for example, a built-in system of an external personal computer, server, programmable logic controller (PLC), or computerized numerical control (CNC) system, connected by a network (LAN). The analysis system may be caused to analyze (calculate) optimal set values (optimal values of gain, rate of increase, and other values) unique to the assist device 1 (that is, unique to the user), and to transmit analysis information containing the optimal set values that are analyzed results (calculated results) to the controller 61 (communication unit 64) of the assist device 1. By analyzing the motion of the user, assist force, and the like, with the analysis system, optimal assist torque obtained in consideration of the type of work (repetition, lift-up level, or the like) or the capability of the user can be output. Based on the analysis information (for example, gain and rate of increase) received from the analysis system, the right and left actuator units adjust their own operations (for example, change the gain and the rate of increase to the received gain and rate of increase).

What is claimed is:

1. An assist device that reduces a load on a lower back of a user by assisting the user with a movement of each thigh of the user relative to the lower back, the assist device comprising:
a body-worn component worn at least around a hip of the user;
a thigh unit mounted on the body-worn component and each thigh of the user, the thigh unit being configured to transmit assist torque for assisting the user with a movement of the thigh relative to the lower back or a movement of the lower back relative to the thigh;
a torque generation unit having an actuator configured to generate the assist torque;
a control box disposed above the hip of the user on the body-worn component, the control box accommodating the torque generation unit and a controller configured to control the torque generation unit; and
a power transmission unit configured to transmit assist torque generated in the control box to the thigh unit.

2. The assist device according to claim 1, wherein the power transmission unit includes a cable and a tension adjustment part configured to adjust tension of the cable.

3. The assist device according to claim 1, wherein:
the body-worn component includes a lower back support part worn around the hip of the user;
the thigh unit includes a right thigh unit worn on a right thigh of the user and a left thigh unit worn on a left thigh of the user;
the right thigh unit is mounted on the lower back support part via a right hip base, the right hip base is connected to a right hip portion of the lower back support part;
the left thigh unit is mounted on the lower back support part via a left hip base, the left hip base is connected to a left hip portion of the lower back support part;
the power transmission unit includes a right frame and a left frame, the right hip base and the control box are connected via the right frame, the left hip base and the control box are connected via the left frame; and
each of the right frame and the left frame has less stiffness in a right and left direction with respect to the user wearing the assist device than stiffness in a front and rear direction with respect to the user, each of the right frame and the left frame is configured to elastically deform so as to curve in the right and left direction for a width of the hip of the user.

4. The assist device according to claim 1, wherein:
the thigh unit includes a thigh worn portion having a surface that contacts with the thigh of the user and a thigh belt wound around the thigh of the user;
the thigh worn portion has a plurality of slits along a thigh extending direction that is a direction in which the thigh of the user extends;
the thigh belt is inserted through the slits; and
a length of the slits in the thigh extending direction is greater than a length of the thigh belt in the thigh extending direction such that the thigh belt inserted through the slits is slidable along the thigh extending direction of the slits.
